(19) United States
(12) Patent Application Publication
(10) Pub. No.: US 2009/0327348 A1
(43) Pub. Date: Dec. 31, 2009

(54) JOB SUPPORT SYSTEM AND ITS METHOD

(76) Inventor: Shigemasa Katayama, Tokyo (JP)

Correspondence Address: Jason H. Vick;Sheridan Ross, PC, Suite # 1200, 1560 Broadway, Denver, CO, 80202, US

(21) Appl. No.: 12/438,802

(22) PCT Filed: Aug. 31, 2007
(86) PCT No.: PCT/JP2007/067052

§ 371 Date: Feb. 25, 2009

(30) Foreign Application Priority Data
Sep. 6, 2006 (JP) 2006-241184

(51) Int. Cl. G06F 17/30

(52) U.S. Cl. 707/104.1; 707/E17.005; 707/E17.044

(57) ABSTRACT

Action frame table part 2 stores a single action frame table consist of a plurality of field items. Label of the field items are respectively worker for the instruction, object person who receives the work, supervisor for the worker, date and hour, and place. The searching part 4 searches for relevant instruction from each instruction when values of field of the action frame table and the detail table are specified. The search control part 7 of the client 11a provides to the server 10 a search formula specifying the field items. The search control part 7 also displays extraction results on the display part 11 in a predetermined format.

### TECHNICAL FIELD

The present invention relates to a work support system, specifically to how to store data.

### BACKGROUND ART

In a work support system, generally, tables are created in response to forms to be displayed or to be printed in each processing. For example, the following system need to be built when a medical system performing the processing shown in FIG. 1.

At first, a form to be used at each processing is determined. For example, the display shown in FIG. 2 is necessary for processing of an appointment performed in step S1. Data is input into such display. With such data, a dedicated table that is configured with similar items to the above shown in FIG. 3 is created.

The use of master data for entry work contributes laborsaving in inputting the above display. For example, data for an item “patient” may be input by selecting from patient master data. This can also be applicable to other items.

Next, a program for performing the subsequent processing is created by recognizing what does the data written in each field item in the table meant to and how the data to be used at the subsequent processing. In this case, a program for processing electronic medical charts for each physician is programmed. Specifically, appointment of each physician for today is displayed on a list (see FIG. 4) in order to support processing for clinical examination. Physicians input the examination results after the examination (FIG. 1 step S2). For input screen of electronic medical charts, the display shown in FIG. 5 as well as a dedicated table therefor are created similar to the processing for appointment performed in step S1 (see FIG. 6). In this way, programs are created for other processing (FIG. 1, steps S3, S4). As a result, a number of programs shown in FIGS. 7A, 7B, 7C and 8 are created while creating programs for performing processing using data input into the table.

### DISCLOSURE OF THE INVENTION

### Problem to be Solved by the Invention

The system construction method described in the above, however, has the following problems. In the above method, basically a table for each display is required. It is, therefore, the more complex the work becomes, the more number of tables need to be created. In addition, programming needs to be performed on recognition of meaning of data at each table and on how to use such data.

Such programs thus created has a lower compatibility. This is because each table is, basically, configured for forms and so on, so that even if the industry is identical, such tables become completely different tables. In addition, that is because the procedure is not always the same even when the form is identical. In other words, it is difficult to commoditize programs even if the industry is identical. Still more, for work supporting systems for different industries, newly established programs are required.

The inventor thought that a labor saving of system design can be achieved once database realizes a high compatibility, so that variety of database structures are reviewed and invents the database structures according to the present invention.

It is an object of the present invention to resolve the above-mentioned problems and to provide a database system capable of achieving labor saving.

### Means for Solving the Problem

1) A program for supporting work comprising:
A) a database storing instructions at each of phases for works, the database being characterized by the following a1) through a3);
a1) all the instructions being specified by one kind of action frame table including plurality of field items,
a2) labels of the field items being at least worker for the instructions, object person receiving works, supervisor for the worker, supervisor for the object person, time and place, and
a3) each of the instructions being specified by the action frame table, the each instruction corresponding to a detail table capable of listing data number without any limitation, the detail table storing operating actions performed by the worker,
B) an extraction program for extracting relevant instruction once a search formula specifying the worker is provided for each of the instructions;
C) a display program for displaying the extracted instruction in a predetermined format;
D) a writing program for generating new instruction by writing data on the action frame table;
E) a correspondence program corresponding to information for specifying display format on each of the instructions; and
F) an instruction master generation program for generating an instruction master in which new instruction generated from the writing program is compared with instruction belong to upstream of the new instruction, an item being recognized as succession of such item when the same data exist in the item, another item being recognized as succession of another item representing duplication of data for the item when the same data exist in another item, and other items except for these defined in the above are recognized as empty items for inputting therein for each time.

Hence, the database is specified by one kind of action frame table and labels of the field items in the action frame table are at least worker for the instructions, object person receiving works, supervisor for the worker, supervisor for the object person, time and place. It is, therefore, not necessary to separately judge who is the worker and the object person for each instruction. In addition, each of the instructions specified by the action frame table corresponds to a detail table capable of listing data number without any limitation. In this way, details of work for the instruction can be listed on the detail table. Further, the extraction program extracts relevant instruction once a search formula specifying the worker is provided for each of the instructions. As a result, instruction for each worker can be extracted. The display program displays the extracted instruction in a predetermined format. Consequently, the extracted instruction can be displayed in a predetermined format. By storing a display format for each instruction, instruction for work can be informed to each worker.

2) A program for supporting work according to the present invention, the program comprising: A) a database storing instructions at each of phases for works, the database being characterized by the following a1) through a3); a1) all the instructions being specified by one kind of action frame table including plurality of field items, a2) the field items being at least worker for the instructions and object person receiving works, and a3) each of the instructions being specified by the action frame table, the each instruction corresponding to a detail table capable of listing data number without any limitation, B) an extraction program for extracting relevant instruction once a search formula specifying the field items is provided for each of the instructions; and C) a display program for displaying the extracted instruction in a predetermined format.

Hence, the instructions are specified by one kind of action frame table and labels of the field items in the action frame table are at least worker for the instructions and object person receiving works. It is, therefore, not necessary to separately judge who is the worker and the person made instruction for each instruction. In addition, each of the instructions specified by the action frame table corresponds to a detail table capable of listing data number without any limitation. In this way, details of work for the instruction can be listed on the detail table. Further, the extraction program extracts relevant instruction once a search formula specifying the field items is provided for each of the instructions. As a result, instruction for each of the field items can be extracted. The display program displays the extracted instruction in a predetermined format. Consequently, the extracted instruction can be displayed in a predetermined format. By storing a display format for each instruction, instruction for work can be informed to each worker.

3) The program for supporting work according to the present invention, wherein instruction for subsequent order linked by the instruction is written on the detail table. In this way, the interlinked status of each instruction in supporting work can be extracted.

4) The program for supporting work according to the present invention, wherein details of work performed by the instruction is written together with the subsequent order on the detail table. In this way, instruction detail can reliably be conveyed.

5) The program for supporting work according to the present invention, wherein the detail table has a region for storing work result on the details of work together with the instruction for subsequent order. In this way, an instruction based on previous result is generated by succession of the work result when subsequent instruction is generated.

6) The program for supporting work according to the present invention further comprising supervisor for the worker, and supervisor for the object person as field items of the action frame table. In this way, it is possible to generate an instruction in which the supervisor for the worker and the supervisor for the object person are recognized as one of worker and subject person in an instruction related to the instruction.

7) The program for supporting work according to the present invention, wherein title for display corresponding to each field item is stored in a correspondence table for each instruction during display of the field items in a predetermined format. In this way, it is possible to name easy-to-understand names for the person who refers to the screen when screen based on the instruction is displayed.

8) The system for supporting work according to the present invention, the system comprising: A) a database storing instructions at each of phases for works, the database being characterized by the following a1) through a3); a1) all the instructions being specified by one kind of action frame table including plurality of field items, a2) the field items being at least worker for the instructions and object person receiving works, and a3) each of the instructions being specified by the action frame table, the each instruction corresponding to a detail table capable of listing data number without any limitation, B) extraction means for extracting relevant instruction once a search formula specifying the field items is provided for each of the instructions; and C) display control means for displaying the extracted instruction on display means in a predetermined format.

9) The database management system according to the present invention, the system comprising: A) a database storing instructions at each of phases for works, the database being characterized by the following a1) through a3); a1) all the instructions being specified by one kind of action frame table including plurality of field items, a2) the field items being at least worker for the instructions and object person receiving works, and a3) each of the instructions being specified by the action frame table, the each instruction corresponding to a detail table capable of listing data number without any limitation.

Hence, the instructions are specified by one kind of action frame table and labels of the field items in the action frame table are at least worker for the instructions and object person receiving works. It is, therefore, not necessary to separately judge who is the worker and the person made instruction for each instruction. In addition, each of the instructions specified by the action frame table corresponds to a detail table capable of listing data number without any limitation. In this way, details of work for the instruction can be listed on the detail table.

10) The method of supporting work using a computer according to the present invention, the method comprising the steps of: storing instructions at each of phases for works and recording data in a database characterized by the following a1) through a3); a1) all the instructions being specified by one kind of action frame table including plurality of field items, a2) the field items being at least worker for the instructions and object person receiving works, and a3) each of the instructions being specified by the action frame table, the each instruction corresponding to a detail table capable of listing data number without any limitation, B) extracting relevant instruction once a search formula specifying the field items being provided for each of the instructions; and C) displaying the extracted instruction in a predetermined format.

Hence, the instructions are specified by one kind of action frame table and labels of the field items in the action frame table are at least worker for the instructions and object person receiving works. It is, therefore, not necessary to separately judge who is the worker and the person made instruction for each instruction. In addition, each of the instructions specified by the action frame table corresponds to a detail table capable of listing data number without any limitation. In this way, details of work for the instruction can be listed on the detail table.

11) The system for supporting work having a first computer storing instructions at each of phases for works and a second computer comprising:
A) the first computer being a database management device storing instructions at each of phases for works, the database being characterized by the following a1) through a3); a1) all the instructions being specified by one kind of action frame table including plurality of field items, a2) the field items being at least worker for the instructions and object person receiving works, a3) each of the instructions being specified by the action frame table, the each instruction corresponding to a detail table capable of listing data number without any limitation, and B) the second computer providing to the first computer a search formula specifying the field items and displaying the extracted instruction in a predetermined format once result of extraction is provided from the first computer.

Hence, the instructions are specified by one kind of action frame table and labels of the field items in the action frame table are at least worker for the instructions and object person receiving works. It is, therefore, not necessary to separately judge who is the worker and the person made instruction for each instruction. In addition, each of the instructions specified by the action frame table corresponds to a detail table capable of listing data number without any limitation. In this way, details of work for the instruction can be listed on the detail table. The second computer provides to the first computer a search formula specifying the field items and displays the extracted instruction in a predetermined format once result of extraction is provided from the first computer. Consequently, the extracted instruction can be displayed in a predetermined format. By storing a display format for each instruction, instruction for work can be informed to each worker.

12) The system for supporting work according to the present invention, wherein the second computer provides to a person who logged in thereto a search formula for the worker when the login process thereto is performed. In this way, instruction for work can be informed to each worker.

Throughout the specification, the term “labels of the field items” refers to names of items in the action frame table, such term corresponds “worker”, “object person” and so forth in the embodiments.

The term “detail table capable of listing data number without any limitation” refers to a table that can store data having a predetermined item numbers without any limitation on its number.

The term “instruction belong to upstream” refers to an instruction that is based on the present instruction in each of instructions. Obviously, there might be cases that such instruction is generated right before and that such instruction become a base of previously generated instruction through another instruction.

The term “work” obviously includes a case that some or all of which is not commercial activities, and the term used in this application also includes cases of volunteer activities, in-home activities such that instruction is provided from parents to children for example.

The term “instruction” generally refer to an instruction that make person to take some actions, but the term further includes report to instruction. For example, it includes reporting for observation result and so on. The term “instruction” is a concept that includes request.

The terms “worker”, “supervisor for worker” and “supervisor for object person” include ones to be personalized. In other words, the terms include ones carrying out processing by their own judgment in response to some instruction(s) to be provided.

### BRIEF DESCRIPTION OF DRAWINGS

FIG. 1 is a schematic diagram illustrating processing performed in a medical system.

FIG. 2 is a diagram showing an example of a screen shot for processing appointment.

FIG. 3 is a diagram showing a dedicated table of a screen shot for processing appointment.

FIG. 4 is a diagram showing a screen shot listing up today's appointment for each physician.

FIG. 5 is a diagram showing input display of an electronic medical chart.

FIG. 6 is a diagram showing a dedicated table for an electronic medical chart.

FIG. 7 is a diagram showing a dedicated table generated by the medical system shown in FIG. 1.

FIG. 8 is a diagram showing a dedicated table generated by the medical system shown in FIG. 1.

FIG. 9 is a functional block diagram of a work support system according to the present invention.

FIG. 10 is diagram illustrating data structure of instructions.

FIG. 11 is a diagram showing an example of hardware structure realizing the server 10 with a CPU.

FIG. 12 is a diagram showing a relationship among each of tables storing data structure of instructions.

FIG. 13 are diagrams illustrating concrete examples of tables.

FIG. 14 are diagrams illustrating concrete examples of action procedure master and display master.

FIG. 15A is a diagram illustrating a concrete example of in-look screen data and FIG. 15B is a diagram illustrating XML data send to the server.

FIG. 16 is a diagram showing an example of hardware structure realizing a client 11a shown in FIG. 9 with a CPU.

FIG. 17 is a processing flowchart performed at a client.

FIG. 18 are display examples at a client.

FIG. 19a is an example of XML data.

FIG. 19b is a processing flowchart performed at the server.

FIG. 20 is a data example of an instruction to be stored in the server.

FIG. 21 is a data example of an instruction master stored in the server.

FIG. 22 is a data example of a new instruction generated from the instruction master.

FIG. 23 is a processing flowchart performed at a client.

FIG. 24 is an example of a table for work performed at a client.

FIG. 25 is an example of a display screen at a client.

FIG. 26 is an example of a display screen at a client.

FIG. 27 is an example of an instruction generated from input result.

FIG. 28 is an example of a newly generated instruction.

FIG. 29 is data of instruction master for generating new instruction.

FIG. 30 are examples of data structure in the case of corresponding instructions between the source of generation and the destination of generation.

FIG. 31 show a comparison between the present invention and a conventional one.

FIG. 32 is a diagram showing an instruction for generating instruction master (original record).

FIG. 33 is a diagram showing an example of instruction master.

FIG. 34 is a flowchart for generating instruction master.

### DESCRIPTION OF REFERENCE NUMERALS

1: work support system
23: CPU
27: memory

### EMBODIMENTS FOR CARRYING OUT THE INVENTION

### 1. Functional Block Diagram

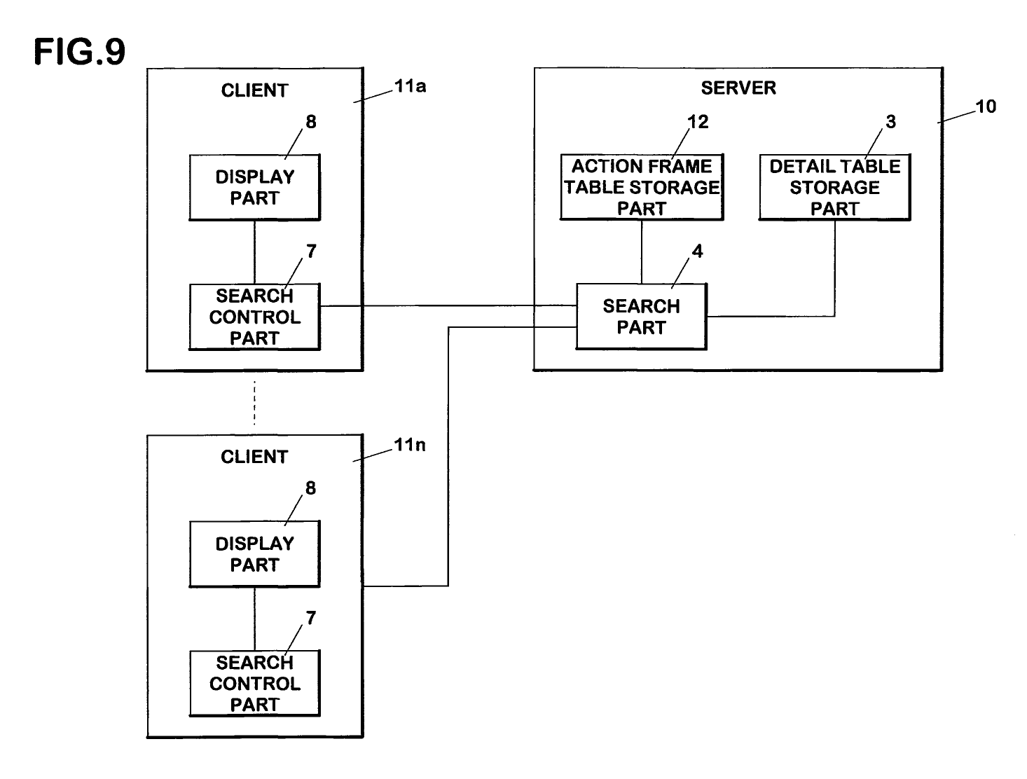

FIG. 9 shows a functional block diagram of a work support system 1 according to the present invention. The work support system 1 comprises a server 10 forming a first computer and clients 11a through 11n severing as a second computer. The server 10 is a database management device storing instructions between a person making commands and worker(s) as a database, and such management device includes action frame table storage means 2, detail table storage means 3 and searching means 4.

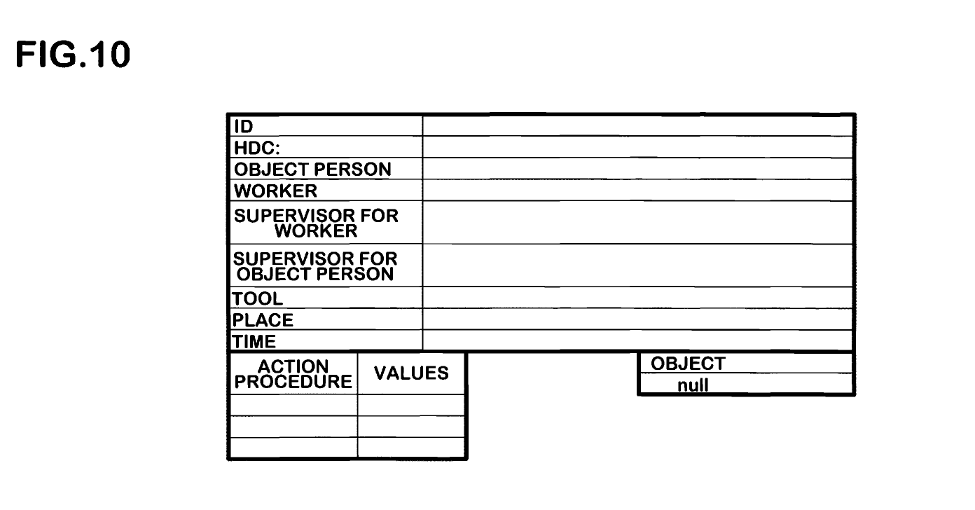

The action frame table storage means 2 is a single action frame table including a plurality of field items, and labels of the field items are respectively worker for the instruction, object person receiving the work, supervisor for the worker, supervisor for the object person, date and hour, and place. With such action frame table, instruction at a specific record is specified. In addition, detailed table corresponds to each instruction. There is no limit on the field numbers in the detailed table and enumeration thereof can be performed. The detail table storage means 3 stores such detailed table therein. In other words, the each instruction is specified by attribution identified with values of the action frame table and the detail table as shown in FIG. 10.

The searching means 4 searches for relevant instruction from each of the instructions when values of field of the action frame table and the detail table are specified. For example, when a request for searching an instruction representing a field “worker” in an action frame table being “ID1000.001” is made, an instruction (record) having such identity can be extracted. This is data which can not be converted in text format and that corresponds to x-ray picture data in a medical system, for example. There are no limits on the number of added data for such object data as well.

The clients 11a through 11n shown in FIG. 9 respectively comprise search control means 7 and display means 8. The search control means 7 provides to the server 10 a search formula specifying the field items. The search control means 7 also displays extraction results on the display means 11 in a predetermined format.

### 2. Hardware Structure

### 2.1 Hardware Structure of Server 10

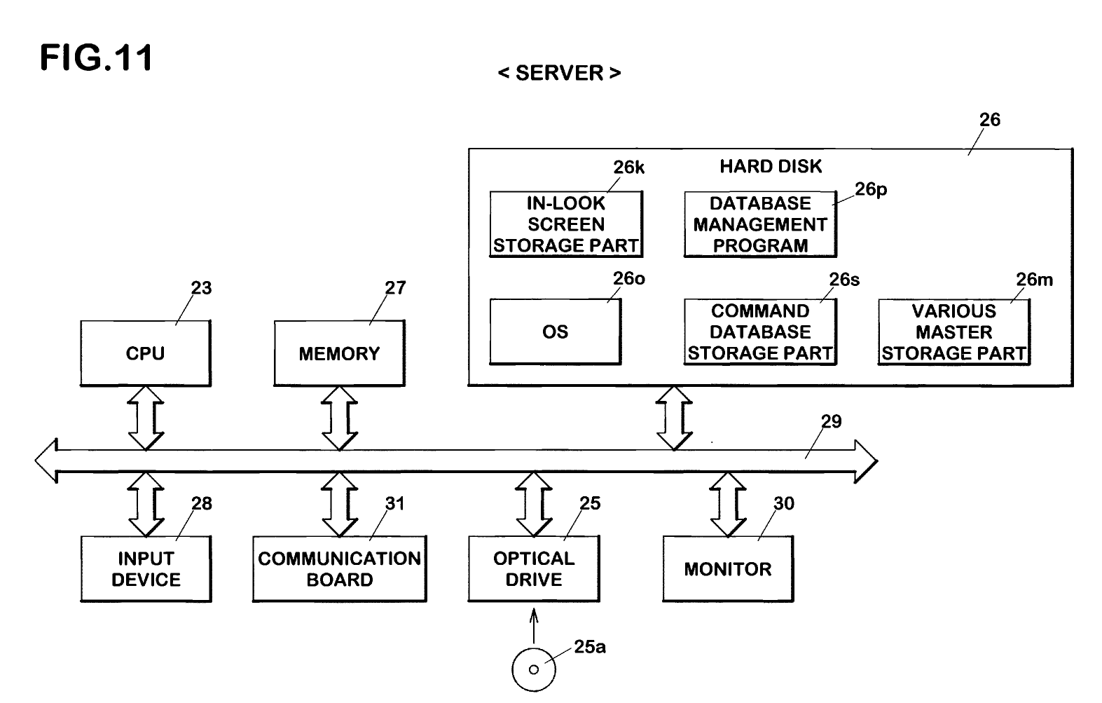

The hardware structure of server 10 in the work support system 1 shown in FIG. 9 will be described using FIG. 11. Such diagram is an exemplary hardware structure using a CPU.

The server 10 comprises a CPU 23, a memory 27, a hard disk 26, a monitor 30, an optical drive 25, an input device 28, a communication board 31 and a bus line 29. The CPU 23 controls each of the parts according to each of programs stored in the hard disk 26 through the bus line 29.

In the hard disk 26, an operating system program 26o (hereinafter referred to as OS for short) and video processing program 26p.

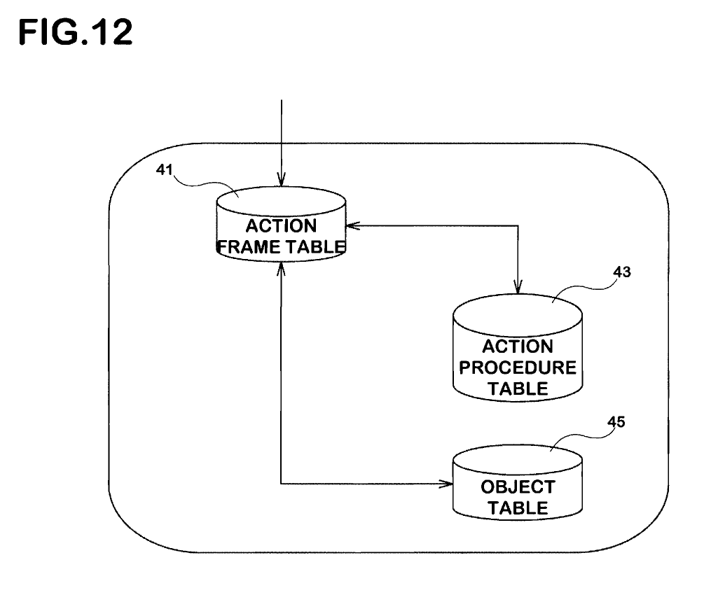

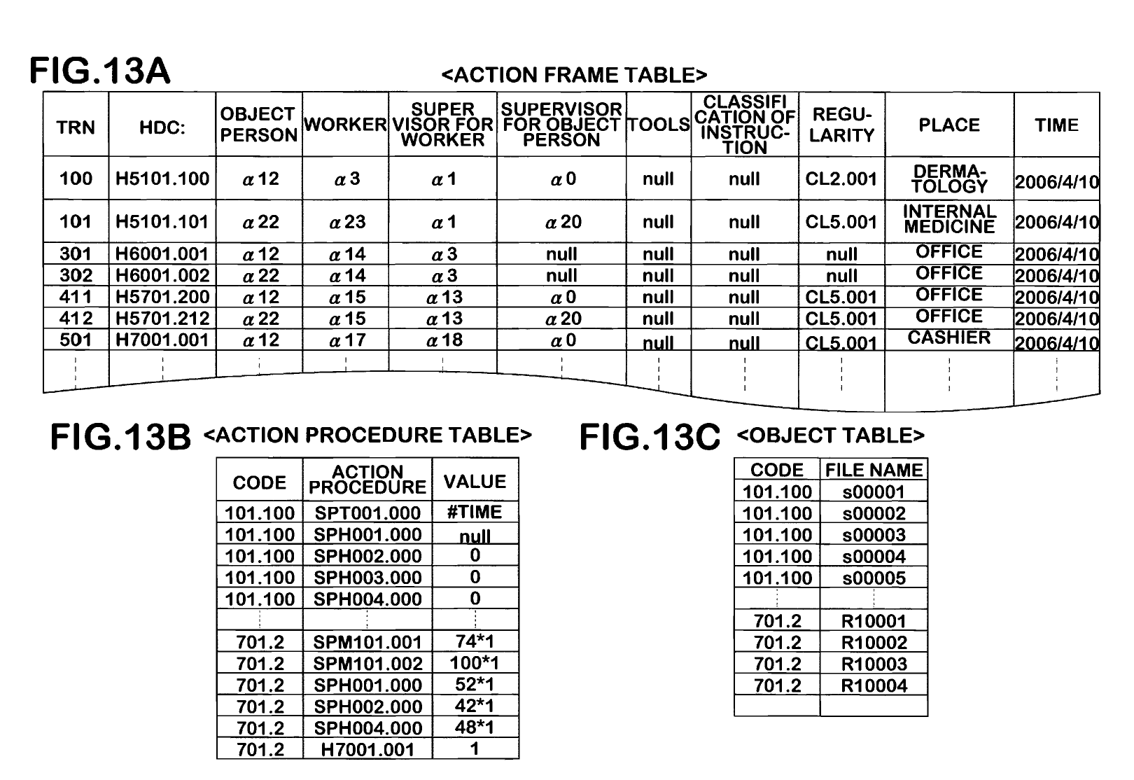

Database management program 26p is a program performing general management of database. In instruction database storage part 26s, each instruction is stored therein in a table structure shown in FIG. 10. For the storage of each instruction in this embodiment, such storage is performed by an action frame table 41, an action procedure table 43 and an object table 45 shown in FIG. 12. FIG. 13 shows exemplary data of the action frame table 41, the action procedure table 43 and the object table 45.

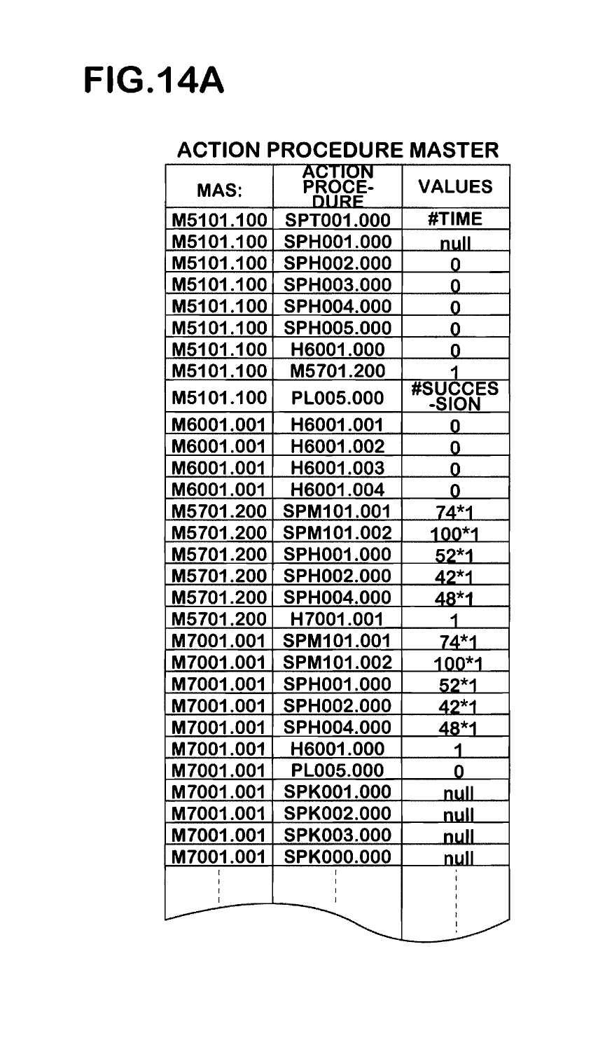

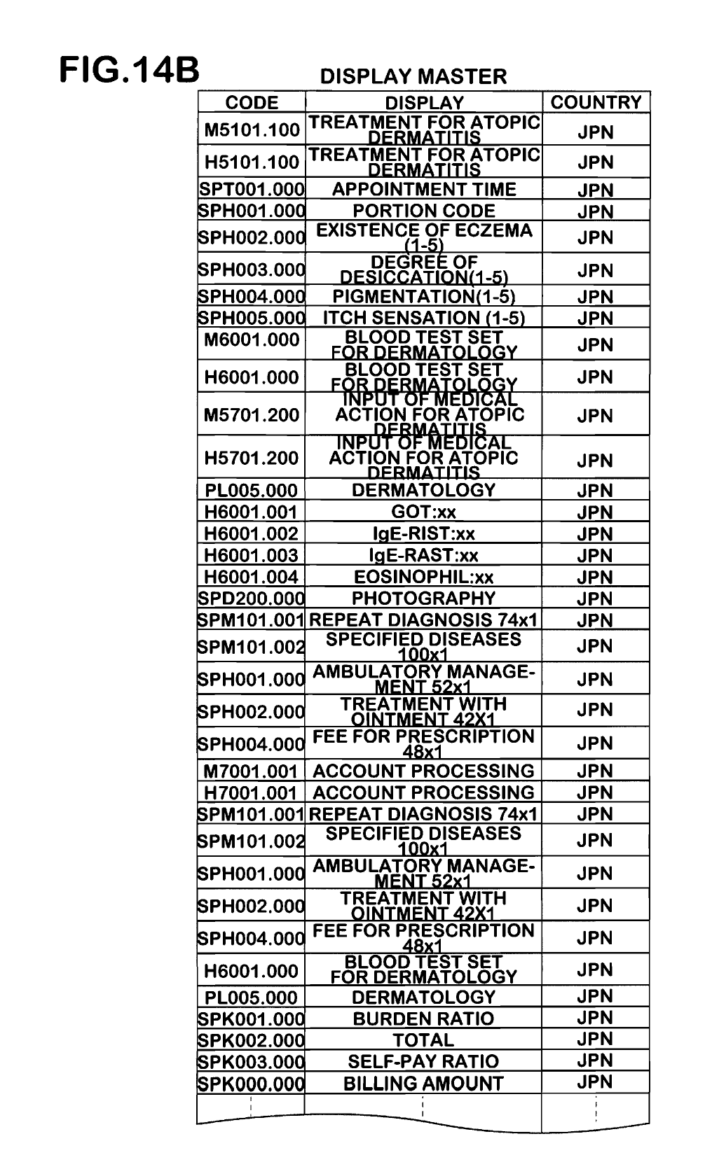

The action frame table 41 includes items such as “TRN”, “HDC”, “object person”, “worker”, “supervisor for worker”, “supervisor for object person”, “tool”, “place” and “time” as fields. “TRN” is an ID of each record. “HDC” is a code for classification and is displayed by referring to a mask for display shown in FIG. 14B. “object person” represents an object person of the instruction. “worker” shows an object person of the instruction. “supervisor for worker” represents a supervisor managing a worker of the instruction. “supervisor for object person” shows a supervisor managing an object person of the instruction. “tool” shows any object used for performing the instruction. Such tool is an X-ray imaging machine, for example. “place” represents a place at which the instruction is performed. For example, the place is “office” and the like. “time” is the time for performance, it can be specified to seconds depending on the system.

There might a case in which items “place” and “tool” are hardly identified. For example, “the first surgery room” may be recognized as both “place” and “tool”. In this embodiment, exclusive relationship is created, that is, when “the first surgery room” is identified for an instruction, it may also be used for other instruction at the same period, it is classified as “place” and it can not be used for other instruction, it is classified as “tool”. Such classification, however, is not necessary absolute rule.

The action procedure table 43 includes “code”, “action procedure” and “values”. The object table 45 comprises “code” and “file name”.

Each of data shown in FIGS. 13A, 13B and 13C are associated with each other and treated as data for one instruction. In this association, the value of “TRN” shown in FIG. 13A would be a key such as relational database (RDB). For instance, for a record in which “TRN” is “101”, action procedures “SPT001.000”, “SPH001.000”, “SPH002.000”, “SPH003.000” and “SPH004.000” to which a code “101.100” matching its initial character series to the values of these being assigned are associated with one another in FIG. 13B, and file names “S00001”, “S00002”, “S00003”, “S00004”, “S00005” to which a code “101.00” shown in FIG. 13C being assigned are associated with one another and treated as one instruction. In this way, each instruction is stored in a form of the table structure shown in FIG. 10.

General master files, such as patient master storing information on patients, physician master recording information on physicians and clerk master storing information on clerks, are stored in various master storage part 26m. In addition, action procedure master and display master shown in FIG. 14 are stored in the various master storage part 26m. The action procedure master is a master for defining action procedure linked at instruction master as described later. The display master is a correspondence table between codes and displays.

Referring back to FIG. 11, in-look screen data for controlling screens used for the case in which the instruction extracted by the server is displayed on each client is stored in in-look screen storage part 26k.

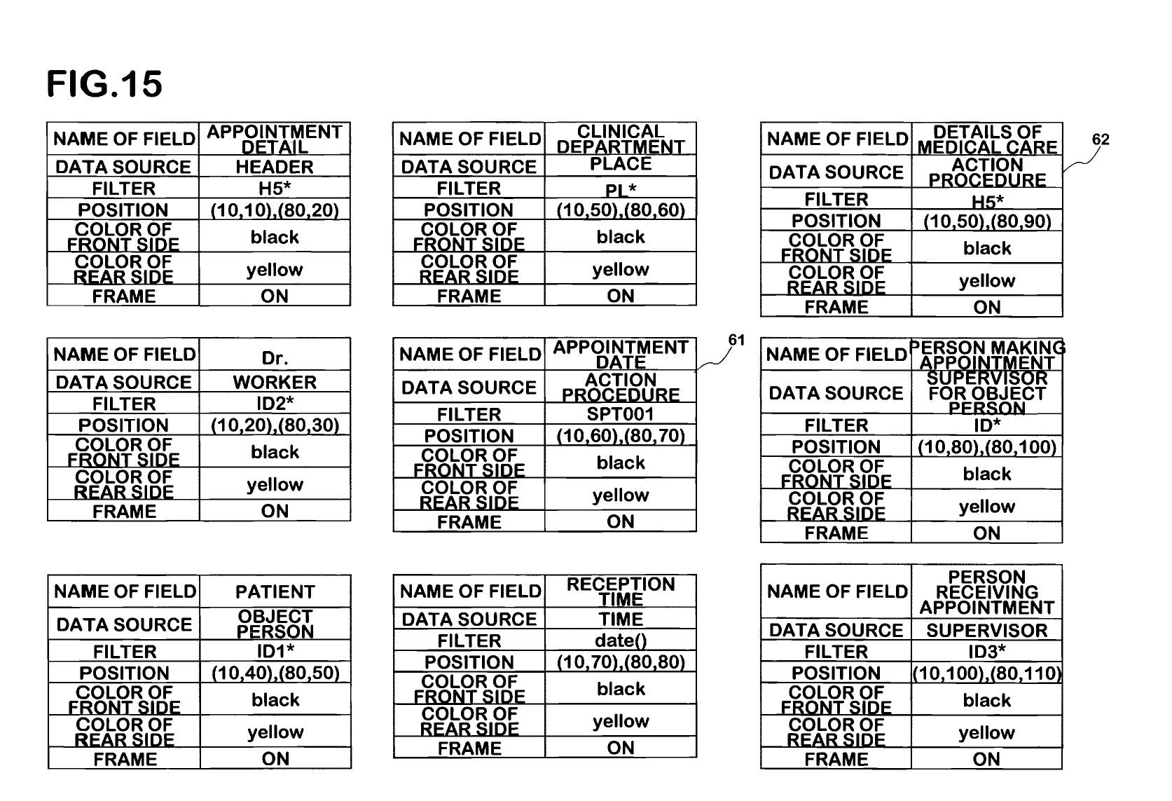

The in-look screen data stored in the in-look screen storage part 26k will be described with reference to FIG. 15. The in-look screen data is data for converting item names to be displayed when instruction of the table structure shown in FIG. 10 is displayed on the clients and that for converting display formats such as display position of box and its color. By converting data during its display, the number of table can be limited to one type while clearly conveying details of the instruction.

An example of in-look data is illustrated in FIG. 15. The “field name” therein is the item name during display of the instruction. “Filter” is a filter using a code capable of being recorded in the field and data entry can not be realized when its feature does not comply with requirement of filter. For example, if the data “H7001” which does not comply with the requirement of a filter “H5*” is stored in item “action frame”, no conversion is performed on the item. In addition, two conversion data 61 and 62 exist in FIG. 15A as data source “action procedure”. In this case, filters “H5*” and “SPT001” are respectively set for the conversion data 61 and the conversion data 62. Thus, these data are sorted and displayed as items “appointment date” and “details medical care” in action procedures using the filter.

Furthermore, conversions of “worker” into “Dr.”, “object person who receives the work” into “patient”, “supervisor for the worker” into “person receiving appointment”, “supervisor for the object person” into “person making appointment”, “date and hour” into “reception time”, “place” into “clinical department” and “action procedure” into “details of medical care” are performed. In addition, “data source” shown in FIG. 15A is item names illustrated in FIG. 10. “position” in the drawing specifies the display position of the item. “color of front side” and “color of rear side” are display colors, “frame” identifies that whether or not a frame is displayed. As will be described later, data on buttons 51 and 52 is omitted in the drawing.

In this embodiment, LINUX (a registered trademark or a trademark) is employed as an operating system program (OS) 26o, any other OS may also be used as well.

Each program is readout from a CD-ROM 25a storing programs and is installed into the hard disk 26. Alternatively, each program can be read out from computer readable medium other than CD-ROM, such as flexible disks (FD), IC cards and soon and be installed into the hard disk. In addition, such program may also be downloaded through a communication line.

In this embodiment, the computer indirectly performs programs stored in the CD-ROM by installing the programs from the CD-ROM into the hard disk 26. However, it is possible to perform the programs stored in the CD-ROM directly from the optical drive 25 without any limitation of the above-described way. Computer implementable programs include not only programs capable of being directly performed by just installing them, but also include the programs need to be converted into other formats and the like (for example, that need to be decompressed from data compression format and the like), in addition to program(s) that can be performed in combination with other module parts.

### 2.2 Hardware Structure of Client

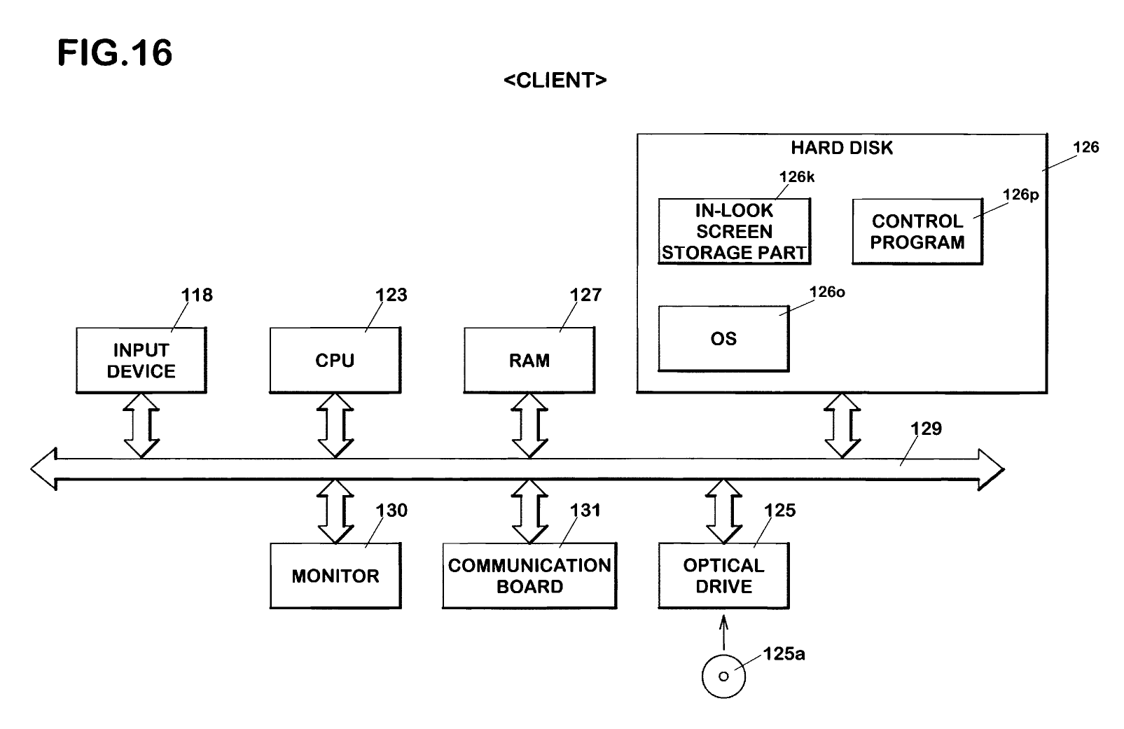

Hardware structure of each client in the work support system 1 shown in FIG. 9 is described with reference to FIG. 16. Such diagram is an exemplary hardware structure configured by utilizing a CPU.

A client 11a comprises a CPU 123, a memory 127, a hard disk 126, a monitor 130, an optical drive 125, an input device 128, a communication board 131 and a bus line 129.

The CPU 123 controls each of the parts according to each of programs stored in the hard disk 126 through the bus line 129.

In the hard disk 126, an operating system program (hereinafter referred to as OS) 126o, a control program 126p and an in-look screen storage part 126k are stored therein.

The control program 126p supports works by extracting instructions from the server 10 and displaying results. Details of such process will be described later. In-look data provided from the server 10 is stored in the in-look screen storage part 126k. In this way, it is not necessary to read out the in-look data in each case.

### 3. Processing with Programs

In what follows, a case in which an appointment for treatment of atopic dermatitis is made to clerk α1 from mother α0 of patient α2, physician α3 conducts a medical treatment on it, clerk α4 inputs such medical activities based on result of the medical treatment, and accountant α5 conducts account processing for that medical activities, will be described as an example. It is assumed that the clerk α1 logs in to the client 11a, the physician α3 logs in to the client 11b, the clerk α4 logs in to the client 11c and the accountant α5 logs in to the client 11d with required IDs and passwords, respectively.

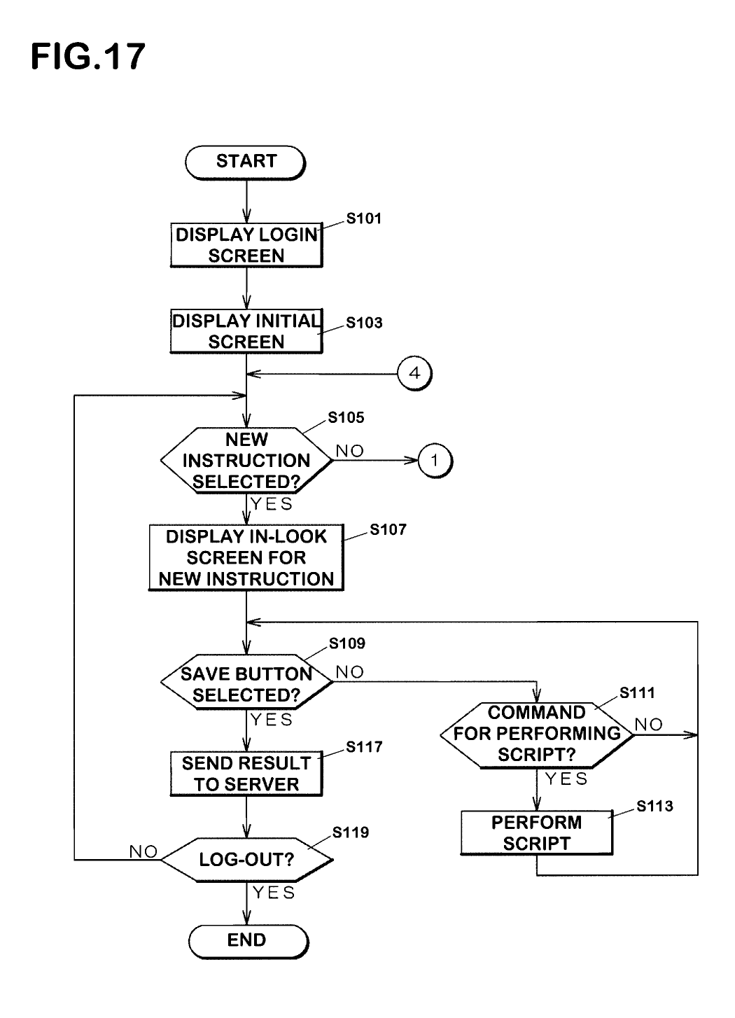

Processing, performed at the client 11a serving as a starting terminal by its CPU 123 according to the control program 126p will be described with reference to FIG. 17.

The CPU 123 of the client 11a displays a login screen (step S101). Subsequently, the clerk α1 logs in to the client 11a with its ID and password. In this way, initial screen (not shown) on which one of “new instruction” and “unprocessed item” can be selected is displayed (step S103).

In this case, since the clerk α1 conducts a new processing for appointment, “new instruction” is selected (step S105) The CPU 123 of the client 11a judges whether or not new instruction is selected (step S105), and an appointment screen stored in the in-look screen storage part 126k is read out and is displayed (step S107) because new instruction is selected in this case.

As already described previously, the in-look screen data stored in the in-look screen storage part 126k is data for converting item names as well as converting display position of boxes and display format such as color and so on as shown in FIG. 15. Data on buttons 51 and 52 is omitted in the drawing.

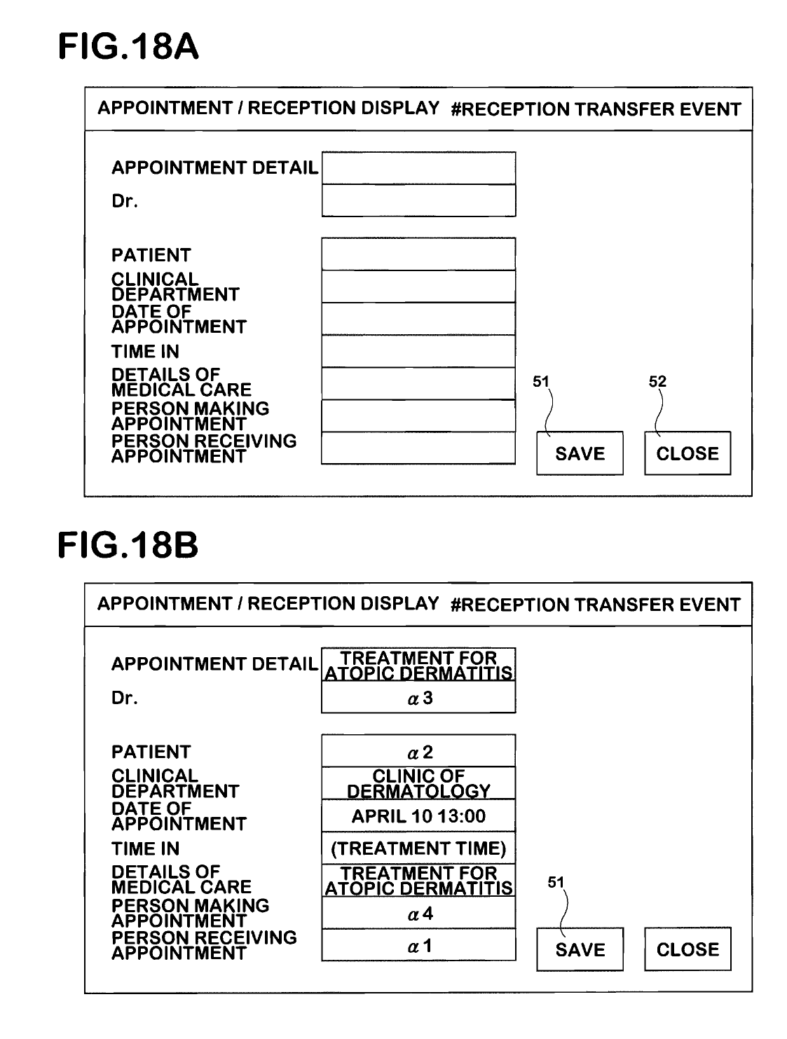

FIG. 18A shows an example of initial screen. By converting display items written in in-look screen data into appropriate items responsive to service on appointment and displaying them, it is apparent to the operator of the client 11a into which item data should be stored.

The clerk α1 inputs to the appointment screen required information such as details of appointment, Dr. and so on. FIG. 18B shows a screen to be displayed upon input of the required information. Upon completion of input on the screen, the clerk α1 clicks save button 51.

The CPU 123 judges whether or not the button 51 is selected (step S109) and it sends the result to the server so that new instruction based on the input is registered (step S117). Consequently, XML data converted into item by item of the server 10 is generated, and the CPU 123 requests the server 10 to store the XML data as a new instruction.

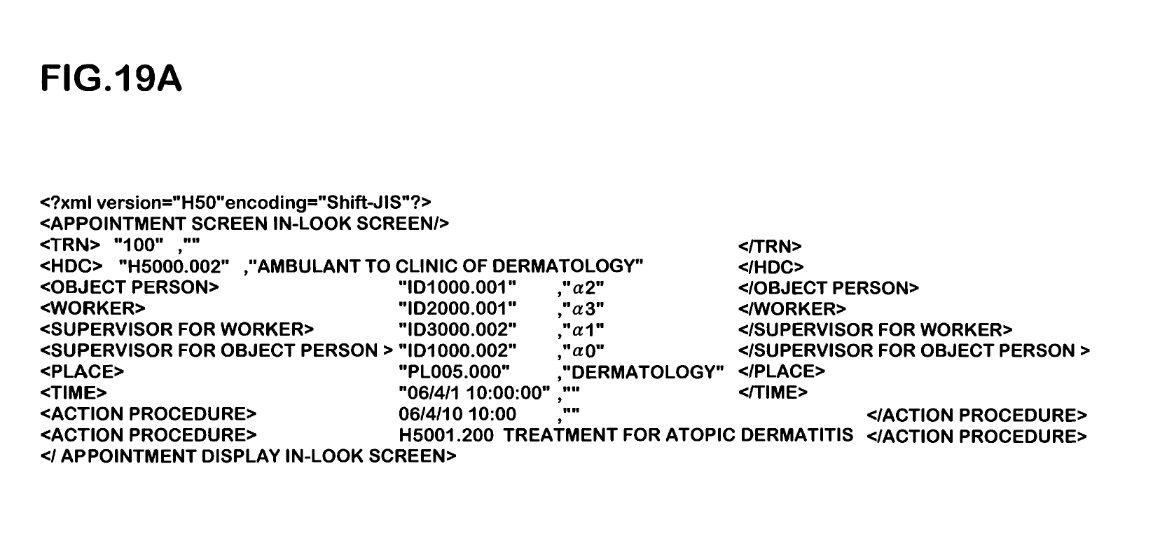

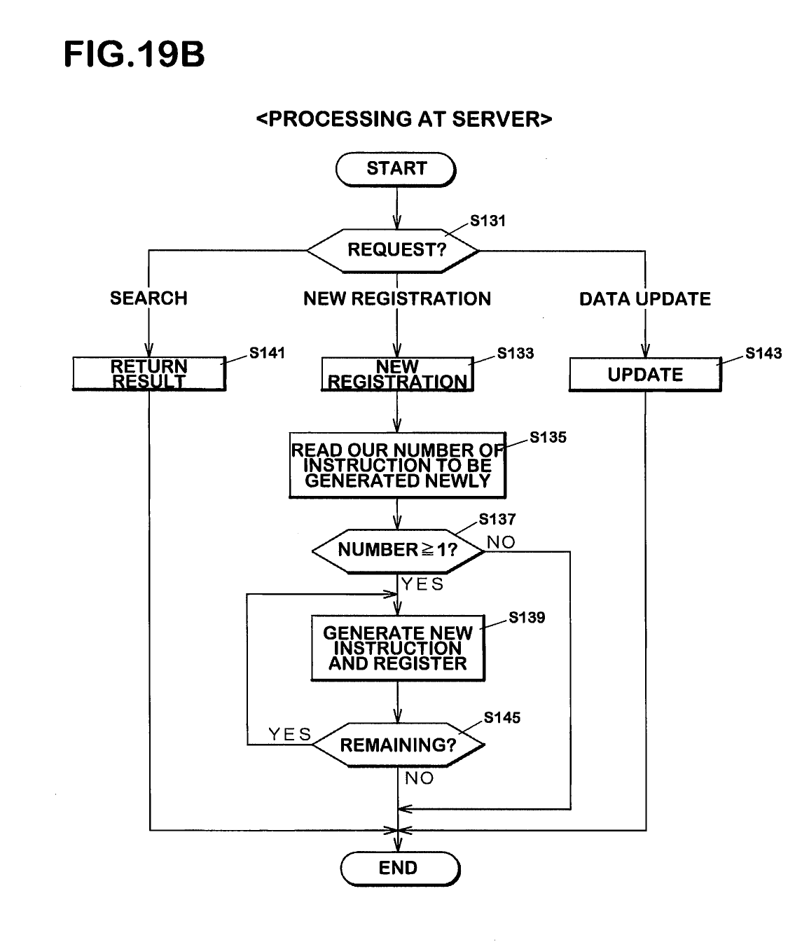

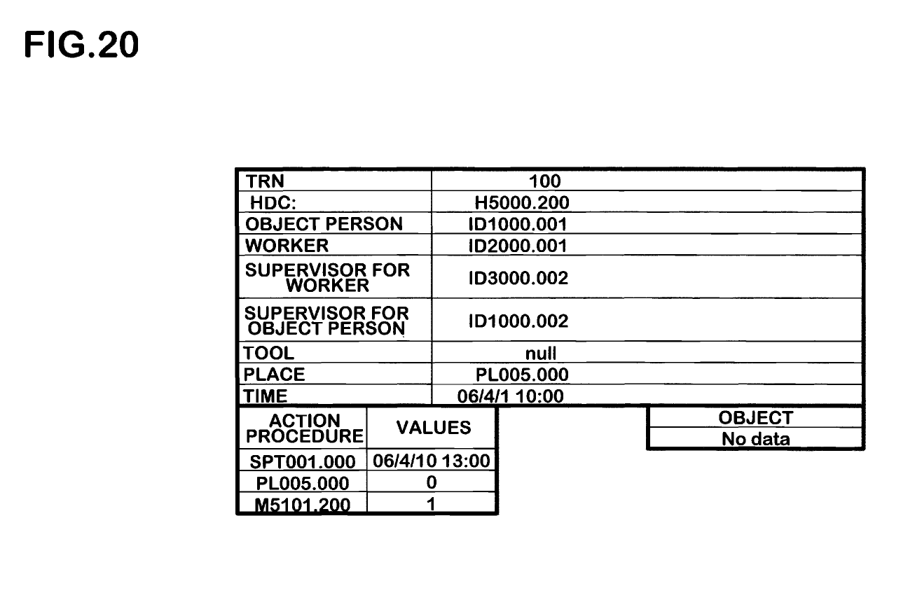

Processing in the case of requesting registration in new instruction will be described with reference to FIG. 19B. There are three processes such as search, new registration and data update for processing performed at the server (step S131, step S133, step S143 and step S141), and each process is almost similar to that performed by ordinary database. Both are differ from each other in that additional instruction is generated when additional instruction can be made during the new registration. Specifically, in that case, if the server receives the XML data shown in FIG. 19A as new instruction together with a request for new registration, such data is added as new entry (FIG. 19B step S133). FIG. 20 shows newly added instructions. Unless otherwise specified, in below description, each of the instruction are shown in the form of gathering data of action frame, of detail table and of object table together as illustrated in the drawing.

The CPU 23 of the server 10 reads out the number of instructions to be generated newly (FIG. 19B step S135). The number of instructions to be generated newly is determined by the number of instruction masters written on “action procedure”. In this case, the number of new instructions to be generated is “1” because just one instruction master “M5101.200” is written is defined as illustrated in FIG. 19B.

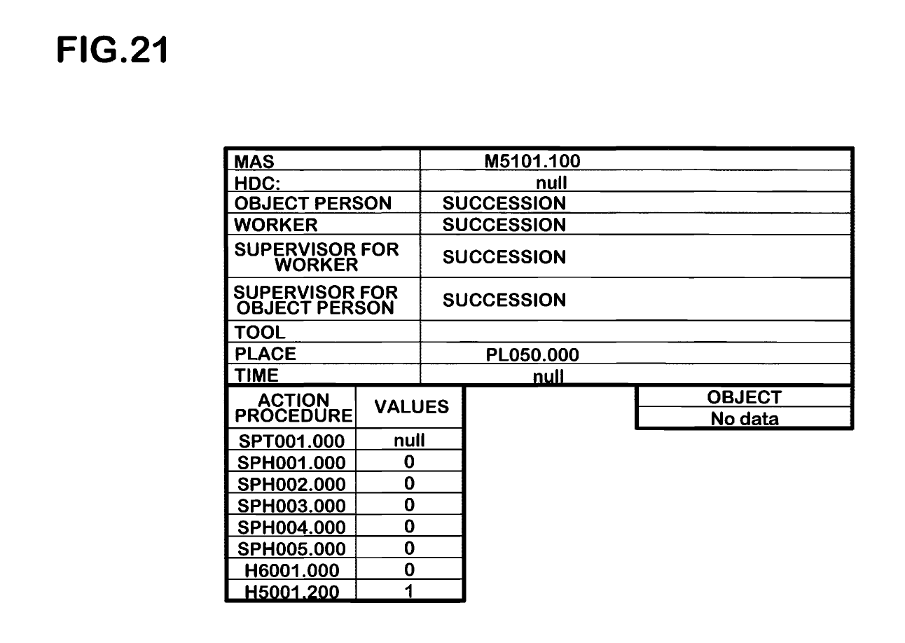

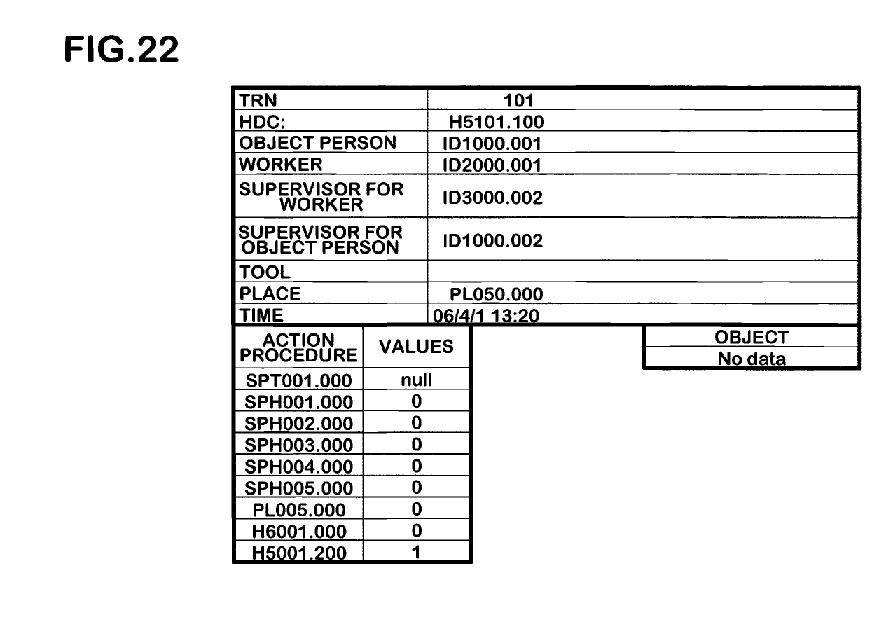

The CPU 23 judges whether or not the number of instructions to be generated newly is equal or more than one (step S137). In this case, since the number is 1, so that instruction to be generated newly is generated and the resultant instruction is registered (step S139). Specifically,” succession” is filled in to the columns “object person”, “worker”, “supervisor for worker”, “supervisor for object person”, “regularity” and “place” in the instruction master shown in FIG. 21. As to these successions, new instruction is generated by reading out corresponding data from the original instruction (see FIG. 18). Consequently, the instructions illustrated in FIG. 22 are generated.

In FIG. 21, its ID is “M5101.100” because it represents instruction master. On the contrary, the ID in FIG. 22 has changed to “H5101.100”. This is to distinguish the instruction master and the instruction generated from the instruction master.

Subsequently, the CPU 23 judges whether or not there exists any remaining of instructions to be generated newly (FIG. 19a step S141). In this case, the process proceeds to the end because no remaining exists.

On the other hand, the CPU 123 of the client 11a judges whether or not log-out is carried out after step S117 shown in FIG. 17 (17 step S119), steps subsequent to step S105 are repeated if no log-out is carried out.

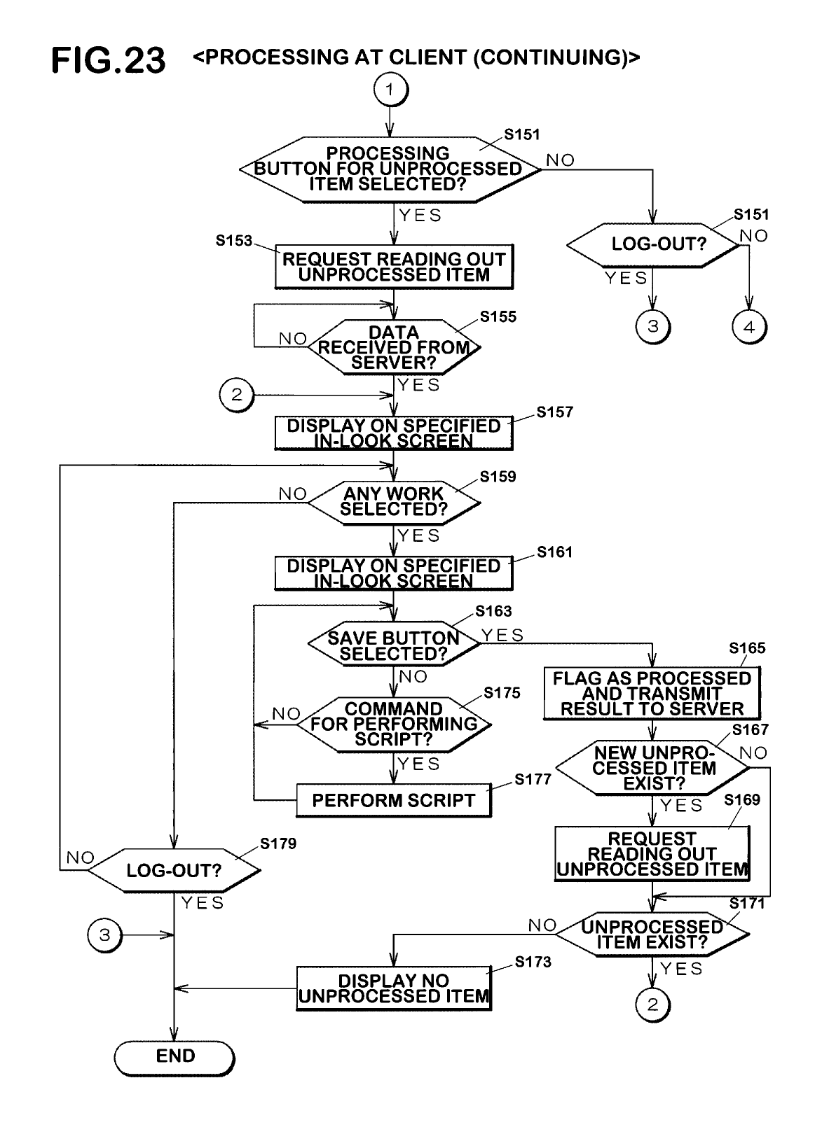

Subsequently, processing performed at the client 11b will be described with reference to FIGS. 17 and 23. The client 11b displays a login screen (FIG. 17 step S101), and the physician α3 logs in to the client 11b with its ID and password. In this way, the CPU 123 of the client 11b displays the initial screen (step S103).

The physician α3 selects a request for reading out unprocessed item after the login. The CPU 123 of the client 11b judges whether or not new instruction is selected (step S105). It is judged whether or not a button for unprocessed item is selected because no new instruction is selected in this case (FIG. 23 step S151).

The CPU 123 of the client 11b requests to the server 10 for conducting a search that extracts an instruction of ID 2000.001, such instruction representing the person having such ID being “physician α3” (step S153).

The server 10 conducts a search for all instructions in which the physician α3 its worker's ID 2000.001 corresponds to item “worker” upon receipt of the search request, and send the result to the client 11b which request the search (FIG. 19B steps S131 and S141).

The client 11b judges whether or not it receives the result from the server 10 (FIG. 23 step S155) and displays each instruction in a specified in-look screen format (steps S131 and S141). This can be done by preparing a desired display format.

Alternatively, the client 11b may acquires the specified in-look screen format from the in-look screen storage part 26k of the server 10 when no specified in-look screen format is stored in the in-look screen storage part 126k.

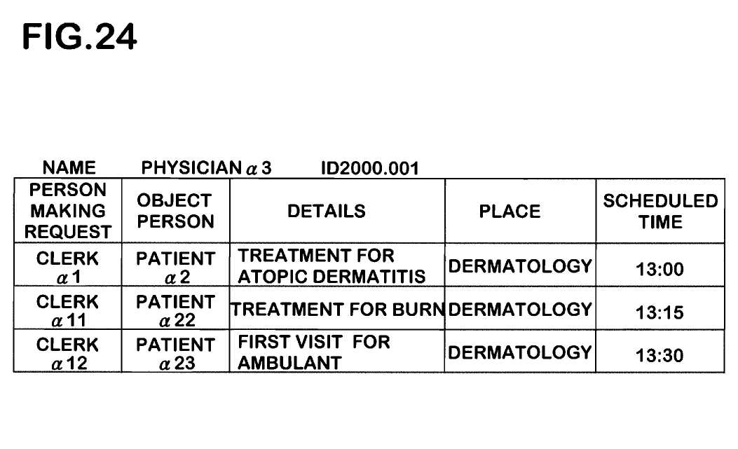

If, there are three instructions to the physician α3, unprocessed items shown in FIG. 24 are displayed. The physician α3 selects one of the instructions. Here, it is assumed that an instruction representing its object person being “patient α2” is selected.

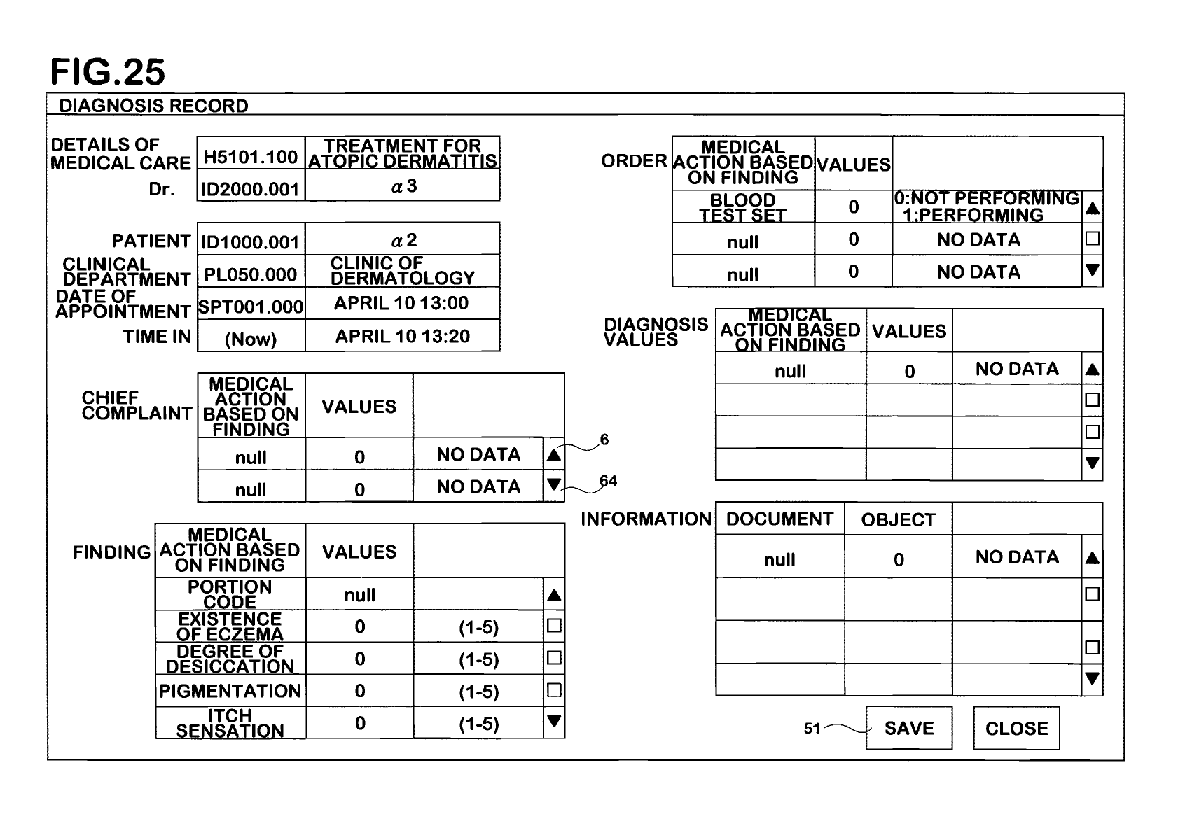

The client 11b of the CPU 123 judges which of the instructions is selected (FIG. 23 step S159) and when any of which is selected, the selected instruction is displayed in a predetermined in-look screen format by reference to a classification code (HDC) (step S161). In this case, the instruction illustrated in FIG. 22 is selected, an in-look screen format to be used is determined from its action frame code “H5101” and a display screen image for input shown in FIG. 25 is displayed.

Also, the client 11b of the CPU 123 judges whether or not the save button 51 is selected (FIG. 23 step S163) and judges whether or not a script performing command exist when the button is not selected (step S175). The CPU 123 to which the script performing command is provided performs such script (step S177). For example, when a button 64 illustrated in FIG. 25 is selected, a script adding one item is performed. Specifically, though two items are displayed, but the number becomes three.

Thus, the physician α3 carries out a predetermined input and the physician α3 selects the save button 51 when such input is completed. The CPU 123 of the client 11b judges whether or not the save button 51 is selected (FIG. 23 step S163). Consequently, the input of such instruction is made to processed ones and the result of which is sent to the server (FIG. 23 step S165).

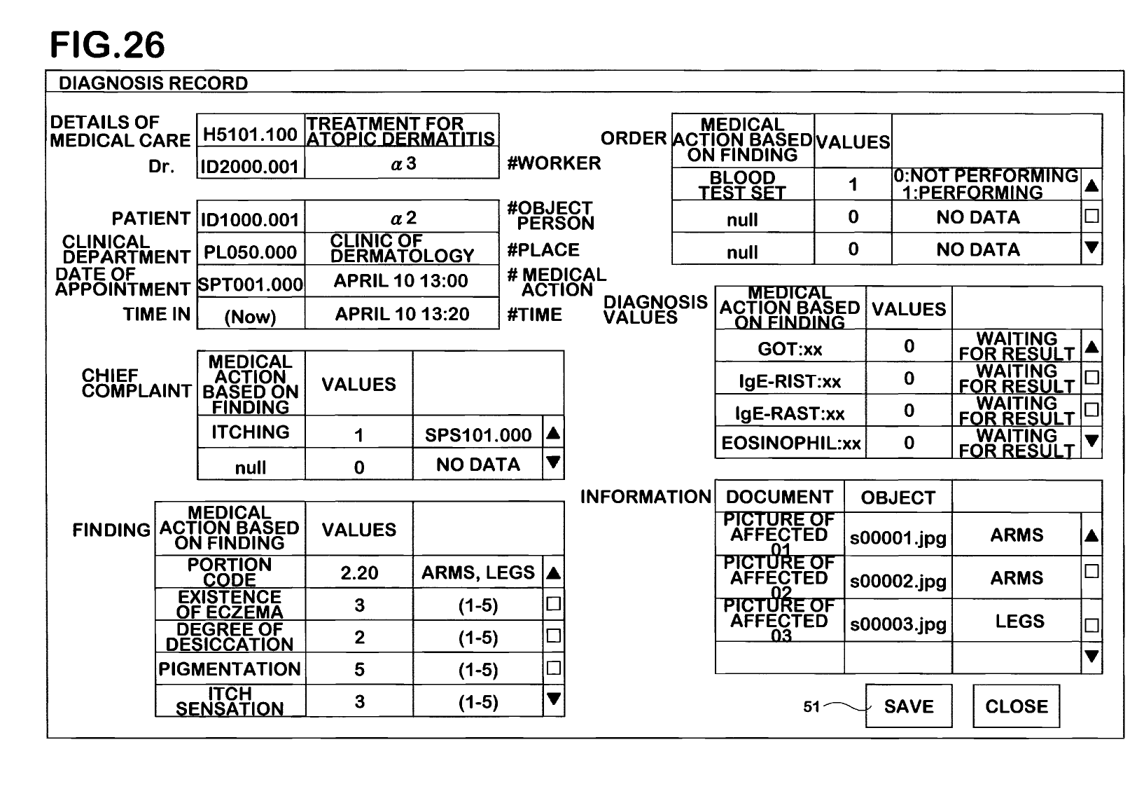

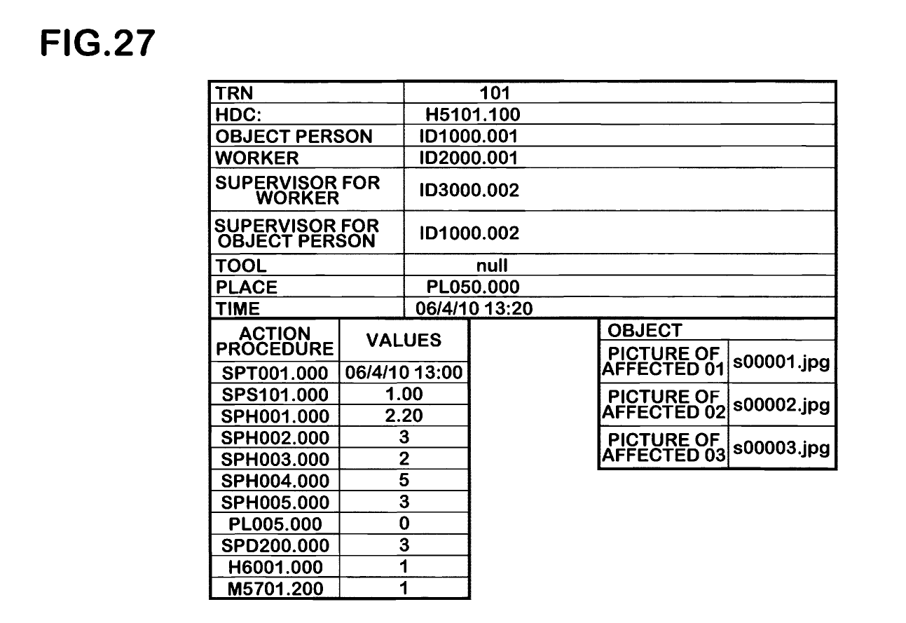

Detailed description of the processing for step S165 is omitted since that is similar to step S117 of FIG. 17. In this way, new instruction is registered in the server. For example, the instruction illustrated in FIG. 27 is registered when the button 51 is selected in the screen shown in FIG. 26.

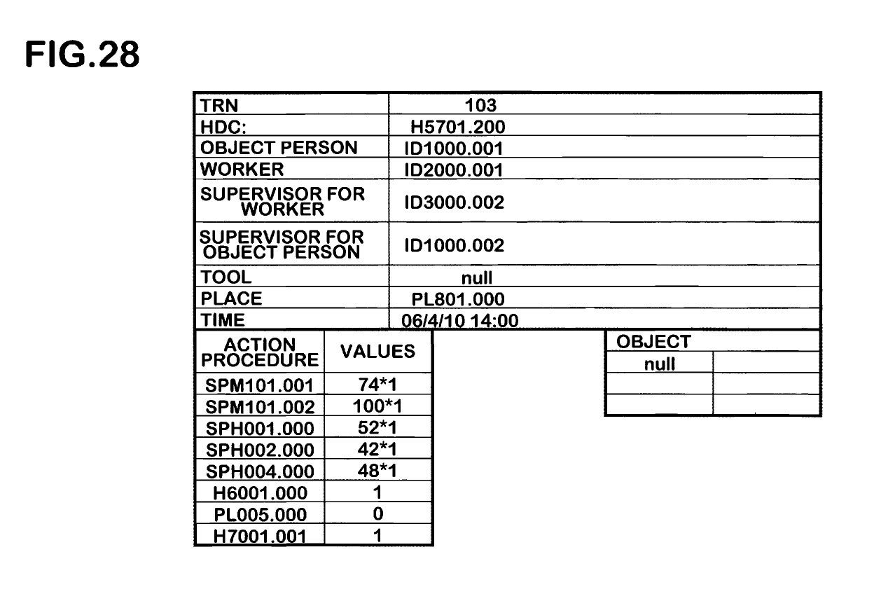

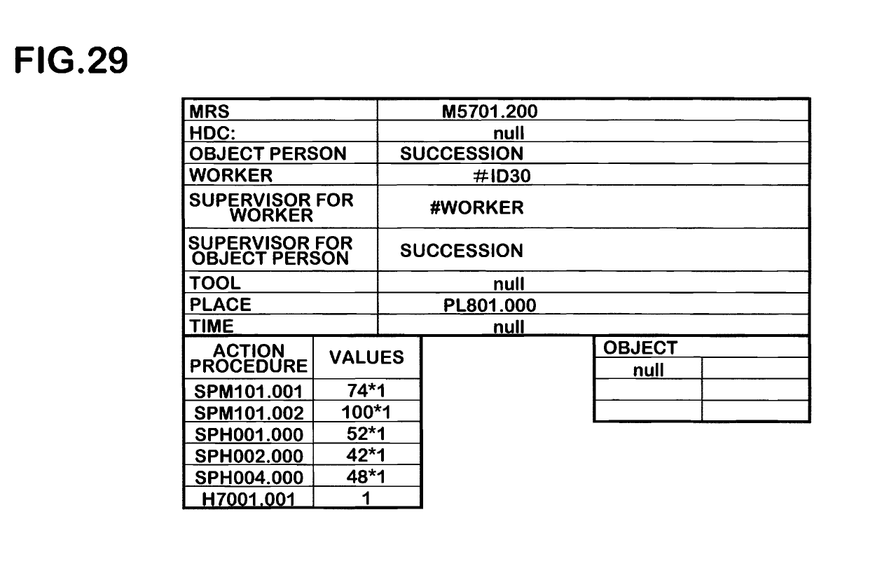

Upon registration of such instruction, one instruction newly generated for “action procedure” is specified. Consequently, the server 10 generates new instruction (FIG. 19, steps S137 through S141). Generation of new instruction is similar to that has been described. The newly generated instruction is shown in FIG. 28. An instruction master, used for the generation of instruction shown in FIG. 28, is illustrated in FIG. 29. In FIG. 29, items “worker” and “supervisor for worker” respectively correspond to items “#ID30” and “#worker”. The former meant to substitute an ID for a person who made login out of people having IDs that start with “ID30” and the latter meant to substitute a value specified as an item “worker” in the instruction that invoke this master. A method of generating such instruction master will be described later.

The client 11b of the CPU 123 judges whether or not new unprocessed item (FIG. 23 step S167), a request for reading out the unprocessed item is made to the server 10 when any exists (step S169). When no new unprocessed item exists, no process of step S169 is performed. It is also judged whether or not unprocessed item exist (step S171), steps subsequent to step S157 are repeated when new unprocessed item exist. On the other hand, a display representing no existence of unprocessed item is displayed when new unprocessed item exist (step S173) and the process ends.

Alternatively, if the physician α3 selects none of work out of the instructions displayed at step S157 and performs log-out processing, the CPU 123 judges that log-out instruction is made (step S179) and the process ends. Similar process is performed when log-out instruction is carried out at step S151.

Instructions will be registered by the clerk a 4 and accountant α5 under the similar way.

As described in the above, instructions within work are focused and uniform attributes such as worker for the instruction, object person who receives the work, supervisor for the worker, supervisor for the object person, date and hour, and place are stored in the database. In this way, it is possible to specify the person who will perform the work for each instruction by just conduct a search with a value “worker=α3”.

The instructions may also be specified with worker for the instruction, object person who receives the work, supervisor for the worker and supervisor for the object person. In this way, it is possible to make contact with the supervisor for the worker during the each work.

Also, in the invention according to the present invention, all the instructions are composed of using a table of single descriptor and a detailed table having no such restriction as described in the above. In this way, a high versatility database having table structure can be built.

In the case of managing instructions by attributions as described in the above, though such management is convenient, details of display on the instruction are hard to understood when such display is made as they are. For such problem, instruction details can be appropriately communicated by preparing a conversion table for display so called in-look screen in response to each of instructions.

In addition, there might be a case in which the way to provide instruction is different even when the work is identical. Even in the case, however, it is possible to obtain a desired work support system by just varying instruction database. Alternatively, an ostensible entirely different system may be obtained by just varying the in-look screen for display of a work support system to the name appropriate for the current work.

Even in the same instruction, the worker may wish to change its display. Such display can be changed by just varying the in-look screen to be used in the case.

The system can not be constructed without performing work analysis in the conventional work support system. On the contrary, the present invention has an advantage of recognizing the entire work in the event by extracting each instruction and accumulating such extraction.

### 4. Other Embodiments

In this embodiment, data of the action frame table and that of the detail table are stored correspondently with transaction ID and code number of the detail table having common first numbers, and a new instruction is generated from the instruction registered at the server 10. As a result, correspondence among these can also be performed by utilizing the correspondence among these data as well as describing IDs for instructions of source of the generation and destination of the generation.

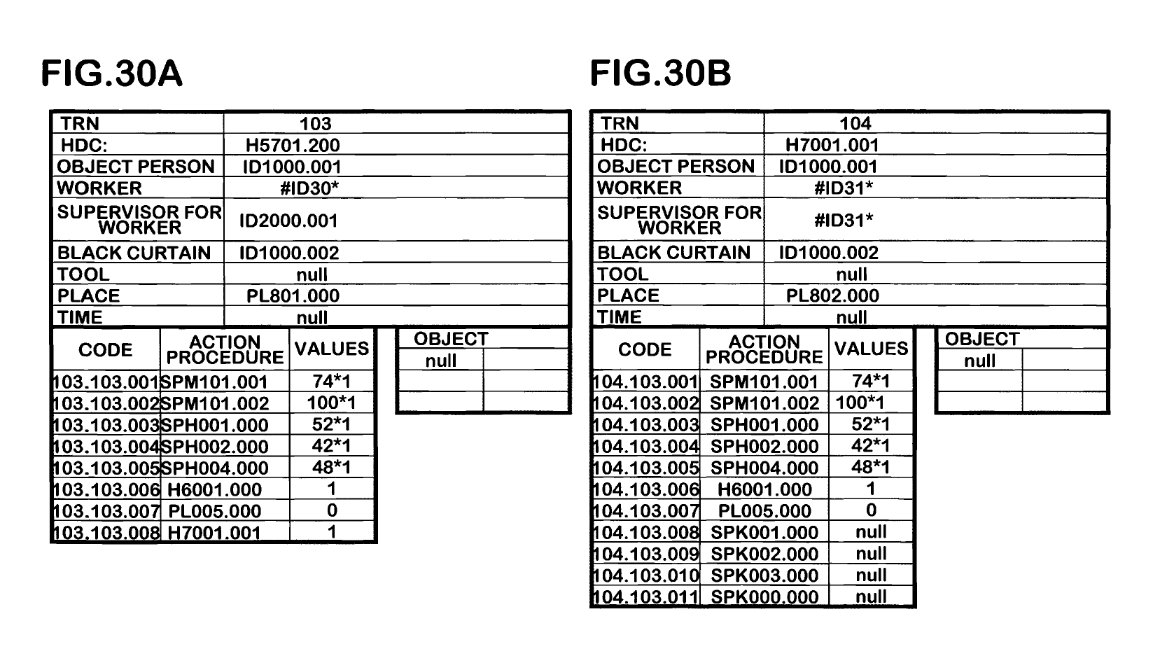

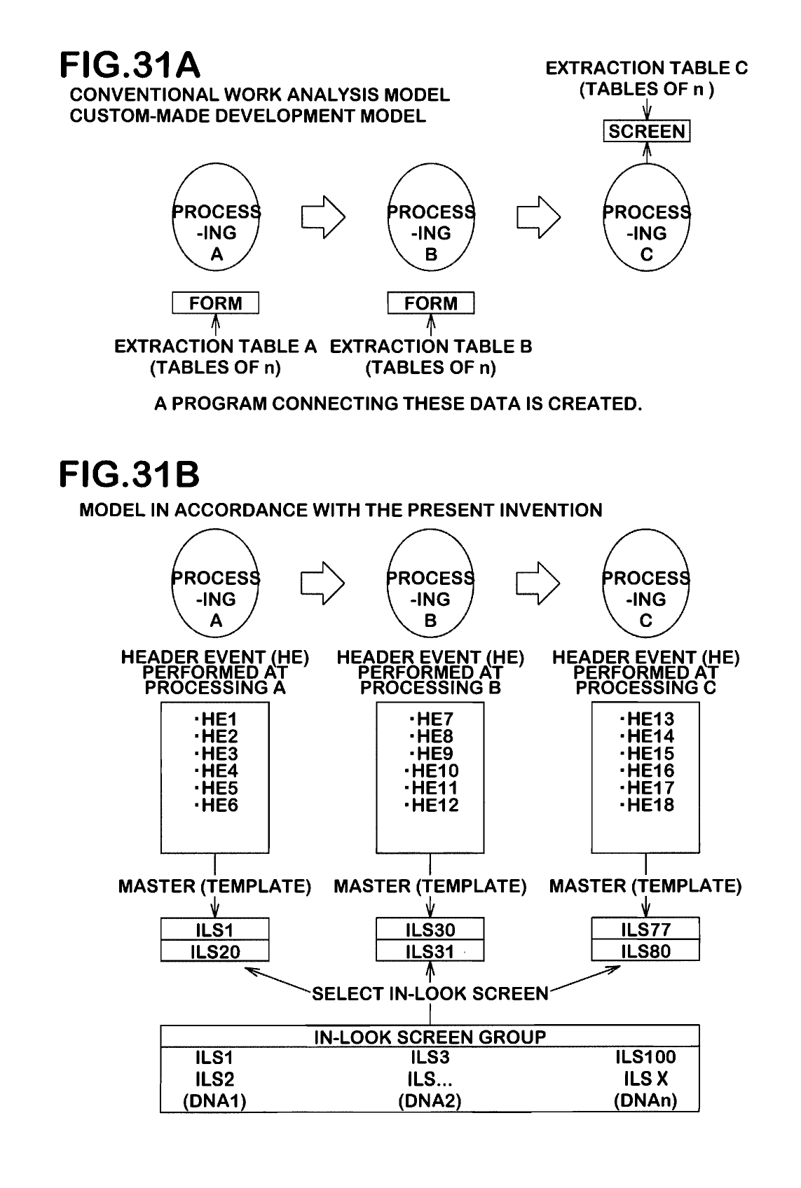

For example, codes “103.103.001, 103.103.002, . . . ” are defined as “action procedure” in FIG. 30A. In this case, it is apparent that the codes are action procedures of transaction “103” and are not the instructions to be source of the generation because of “103.103.001, . . . ”. On the other hand, the codes shown in FIG. 31B are defined as “104.103.001, 104.103.002 . . . ” and a reaction procedures of transaction “104”. It is apparent that the instruction that become source of the generation is “103” because the first numbers are “104”, but right after such numbers is defined as “103”.

Hence, it is possible to judge whether it is generated automatically or not by providing IDs of instructions that become source of the generation to the codes of the detail table. Because of such correspondence, adjustment of one value allows the other value to be adjusted.

As a program to be generated, the control program containing a program for displaying initial screen, a program for carrying out data communication with the server, a program for displaying instructions extracted using in-look screen data, can be adopted to another medical system as it is. The instruction database itself can be adopted as it, if instructions are the same. In that case, such adoption may be made by generating additional instruction(s) and adjusting part of the instructions.

In addition, a work support system can readily be constructed even in entirely different work by varying in-look screen stored in the in-look screen storage part, changing master files of various master storage parts and generating instruction for the work.

Each of instructions itself is built as know-how in one work. For example, assuming that a physician creates an electronic medical chart on which a description is made such as “the degree of the swelling of xxx1, xxx2, xxx3 parts has been examined” as action procedure for yyy disease, using a work support system at a medical institution. By storing such action procedure for yyy disease into an instruction master of appointment for the yyy disease, even a less-experienced physician never overlooks the action procedure by referring such action procedure for yyy disease during the medical diagnosis for a first visit patient for yyy disease.

Consequently, even a person unfamiliar with his/her work may clearly recognize their duty that needs to be processed by accumulating instructions made at a phase as instruction master.

Thus, support of work can be carried out by accumulating instructions because the accumulated data themselves represent details of instructions themselves for the person who specifies worker and object person for the work in this embodiment. Specifically, each of the instructions can be generated even in different work systems by creating a data management program at the server, programs shown in FIGS. 17 and 23 at the clients, and a desired work-supporting program can be provided by creating in-look screen that read out the instructions.

The distinction between the work support system according to the present invention and a conventional work support system will be described with reference to FIG. 31. As shown in FIG. 31A, the conventional system needs to create a table for forms in each instruction. In addition, it requires to recognize meaning of data in each table and to perform programming how such data is used. On the other hand, as shown in FIG. 31B, in the system according to the present invention, the instruction provided at each processing can be stored in the form of action frame table in which its table structure is fixed except for a item “what” within the items, with focusing on attributes (such as, who=“worker”, by whose instruction=“supervisor for worker”, to whom=“object person”, “supervisor for object person”, what=“action procedure”, when=“time”, where=“place”, with what=“tool”). However, since the item “what” is difficult to express with a single item, such item is stored in a detail table having different unrestricted number. Also, as to the item “what”, the detail table is constructed so that it can store values for each of action procedures because its details need to convey at instruction performed continuously from this above work.

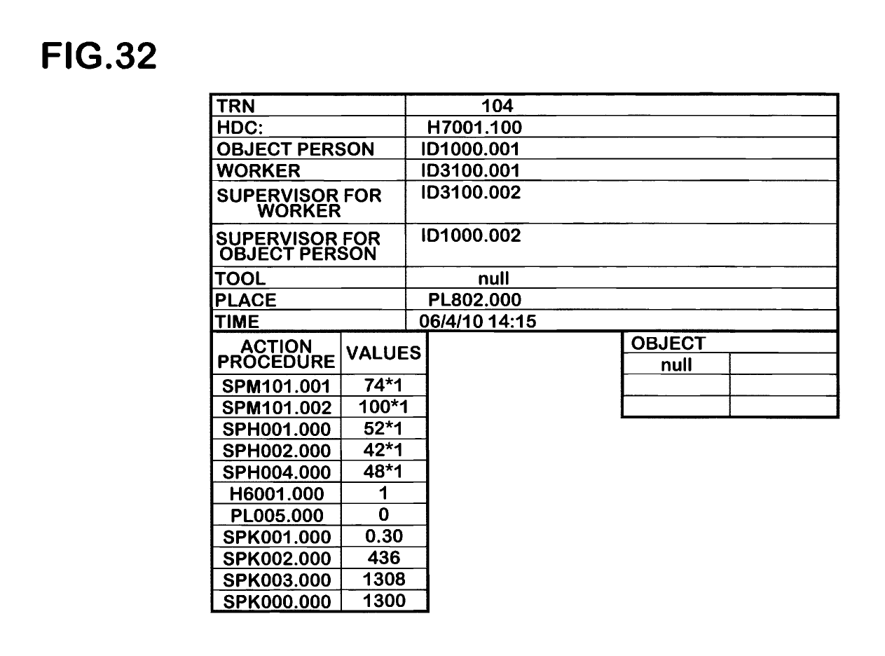

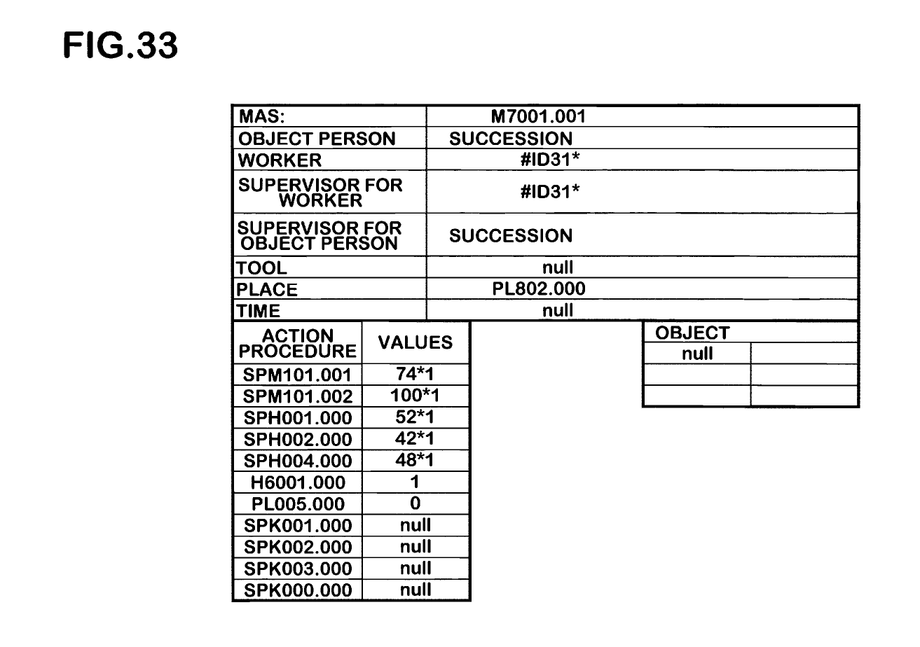

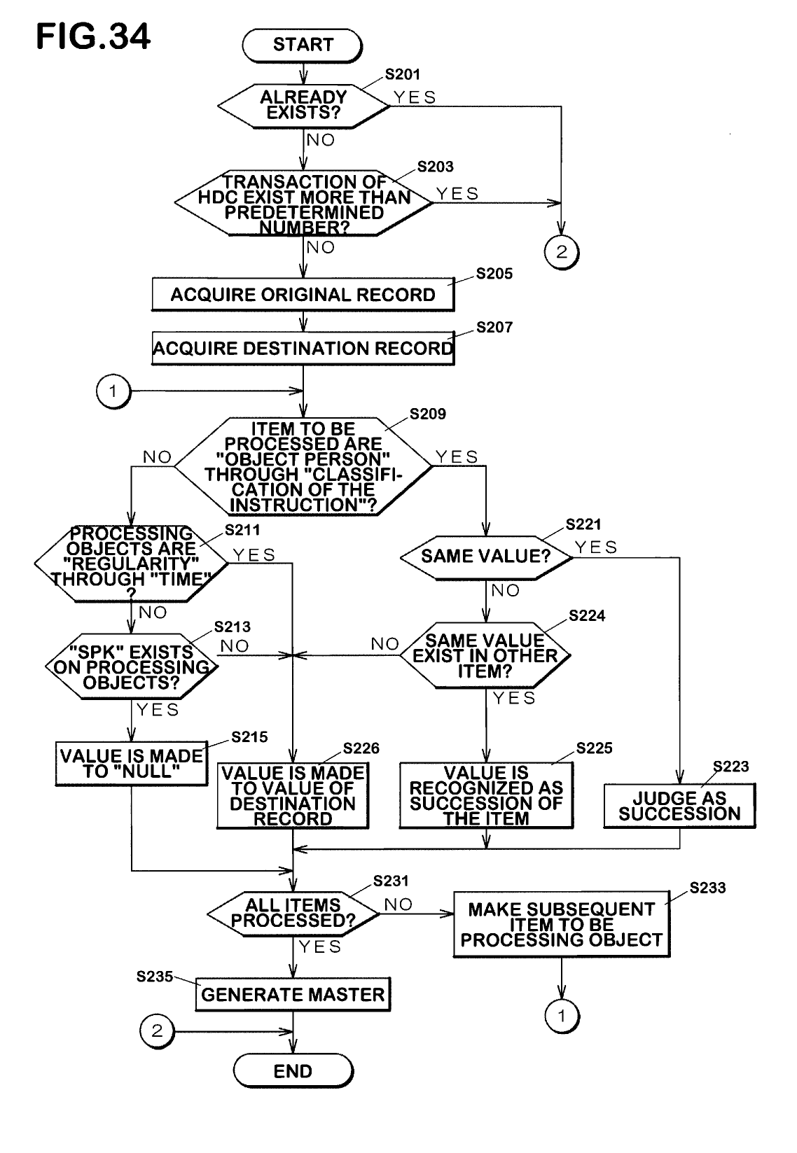

Description is made to the case in which instruction master has already existed in this embodiment. The instruction master may be generated automatically. For example, when the instruction illustrated in FIG. 32 is generated from the instruction shown in FIG. 29, instruction master illustrated in FIG. 33 can be generated. Such processing will be described in FIG. 34. When the instruction illustrated in FIG. 32 is generated from the instruction shown in FIG. 29, the former is referred to as original record and the latter is referred to as destination record.

The CPU 23 of the server 10 judges whether or not instruction master having the same value to numeric portion other than “H” out of HDC of the destination record exists (step S201). In this case, since the numeric portion other than “H” is “7001.001”, it judges whether or not instruction master “M7001.001” having “7001.001” exists.

In the case of existence of the instruction master having the same value, the CPU 23 ends the process because of no necessary of creating such master. When no instruction master having the same value is found, then the CPU 23 judges whether or not transaction of the same classification code for more than a predetermined number exist (step S203). This is because there are many cases that do not have to make as master when such master is not created repeatedly. To set the desired number in appropriate manner, the master is generated only when there are instructions more than the predetermined number.

There are instructions more than the predetermined number in step S203, original record and destination record are fetched (steps S205 and S207). In this case, the instruction illustrated in FIG. 29 is read out as original record and the instruction shown in FIG. 32 is read out as destination record. The CPU 23 judges whether or not items to be processed are “object person” through “classification of the instruction” (step S209). When the CPU 23 judges that the items to be processed are “object person” through “classification of the instruction”, it further judges whether not the value of the original record and that of the destination record are the same (step S221). When the value of the original record and that of the destination record are the same, such record is judged as “succession” (step S223). Then, it is judged whether or not all the items have been processed (step S231), make subsequent item to be a processing object when not all the items have been processed (step S233) and then steps subsequent to step S209 are repeated. If, it is judged that the value of the original record and that of the destination record are not the same in step S221, then it is also judged whether or not the same value exists in other items (step S224), and if any item having the same value exists, the value is recognized to succession of such item (step S225), and the value of which is made to that of destination record if no such item having the same value exists (step S226). As a result, the master that makes its value to the value of destination item can be generated except for succession. In this way, conversion process for the items “object person” through “classification of the instruction” has been completed.

If it is judged that processing objects are not “object person” through “classification of the instruction” in step S209, then another judgment whether or not processing objects are “regularity” through “time” is made (step S211). The value of which is made to that of destination record if it is judged that the processing objects are “regularity” through “time” (step S226). This is because there are many cases that these items have fixed values. On the other hand, if it is judged that the processing objects are not “regularity” through “time” in step S211, then another judgment whether or not “SPK” exists on the beginning of codes of the processing objects (step S213). If “SPK” exists on the beginning of codes of the processing objects, the value becomes “null” (step S215). On the other hand, if the “SPK” does not exist in the beginning, the value of which is made to that of destination record (step S226). Hereinafter, it is judged whether or not all the items have been processed in step S231, if they have not been completed, then steps subsequent to step S209 are repeated. Consequently, in the items where “SPK” exist on the head of the value of the destination record, “null” become their values except for the items “SPK” exist on the head within the action procedure. The CPU 23 generates instruction master once the process for all the items have been completed (step S235). In this case, the instruction master illustrated in FIG. 33 is generated.

Alternatively, the instruction master thus generated may be made to adjustable while displaying thereof before registration as a master.

There is no description on the case in which plural instructions are generated from a single instruction, in other words, no branching has been described in this embodiment. However, the present invention can be used for branching if instructions are stored similarly.

In the above-described embodiment, a dedicated program is stored in each client, it is possible to store just a general-purpose browser and converting into a predetermined at the server and send the file to client for display. Data processing at each client may be carried out a program runs on the general-purpose browser such as Java (registered trademark or trademark). In this case, in-look screen may be fixed for each instruction and can be selected depending on a login ID.

The server, on the contrary, may be an ordinary database. In this case, for performing the processing for generating another instruction based on one instruction (FIG. 19B, steps S135 through S141), request for making new registration may be made to the server from the client.

In this embodiment, items such as classification of the instruction and regularity are used in the action frame table storage part, such usage leave to option.

The present invention has been described in a viewpoint focusing on instructions in a work support system in the above-described embodiment, the present invention may also be understood as a concept specifying human behaviors realizing between humans under the viewpoint above from another aspect. Parameters according to this case may be referred to as behavior model descriptor. In this way, it is possible to compile as a database in a single data form by specifying each instruction with the behavior model descriptor.

In this case, a human being is not a human being itself but it includes a personalized object, i.e. an object capable of being operated in the self-sustained manner based on instruction to be received. In other words, the human behaviors include activities carried out by a personalized subject capable of actively processing.

In the above-described embodiment, XML data using tags is employed for requesting new registration, there is no restriction on communication of data. For example, data communication may be performed in CSV format that is carried out in proper order in order to specify each data belongs to which item.

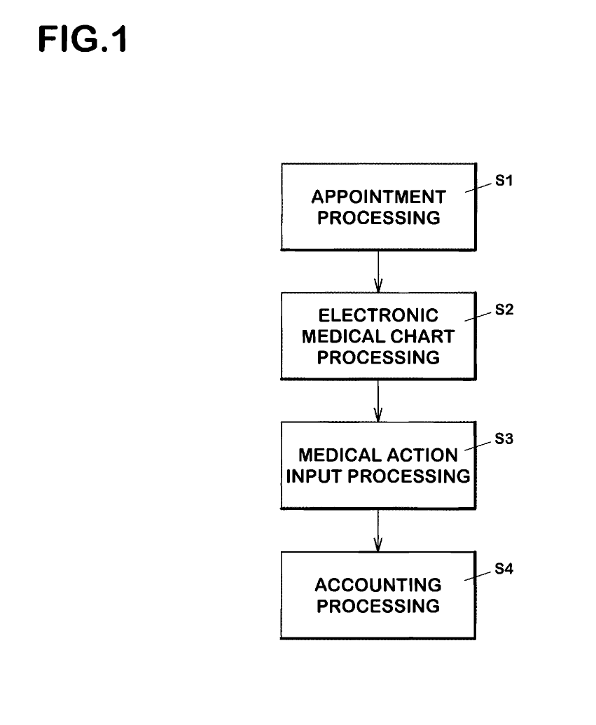

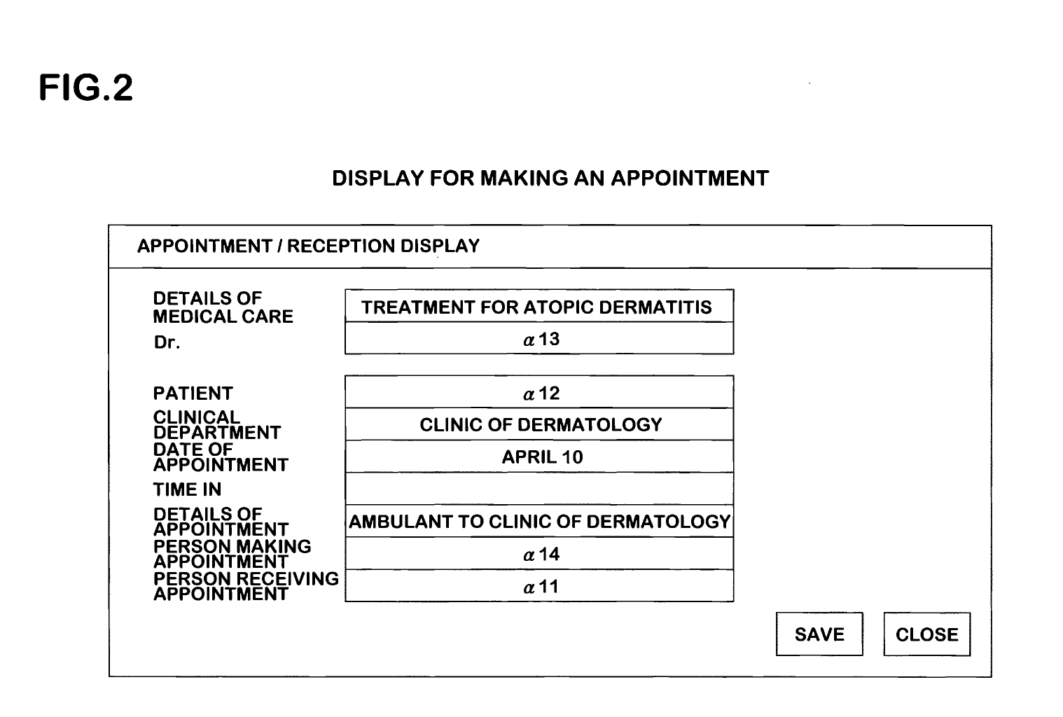

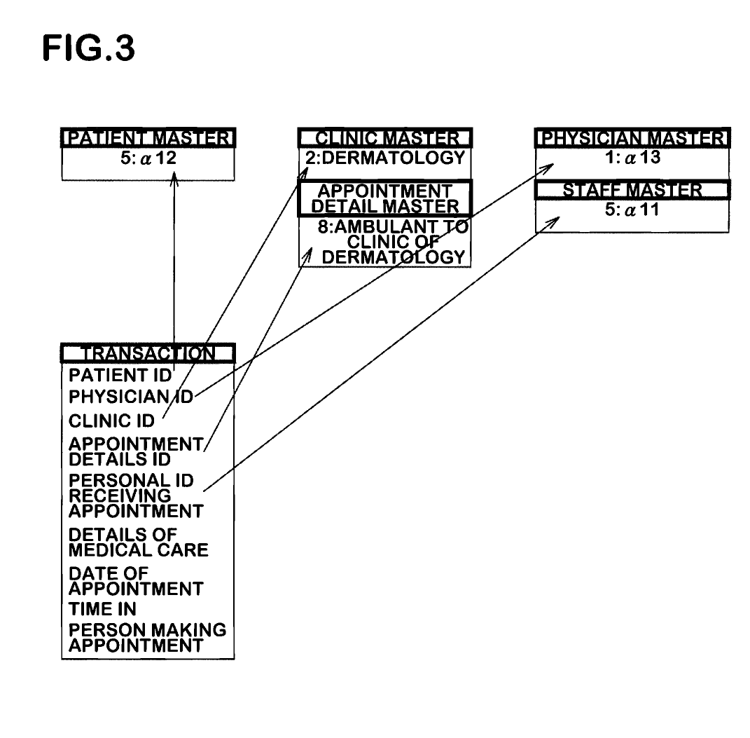

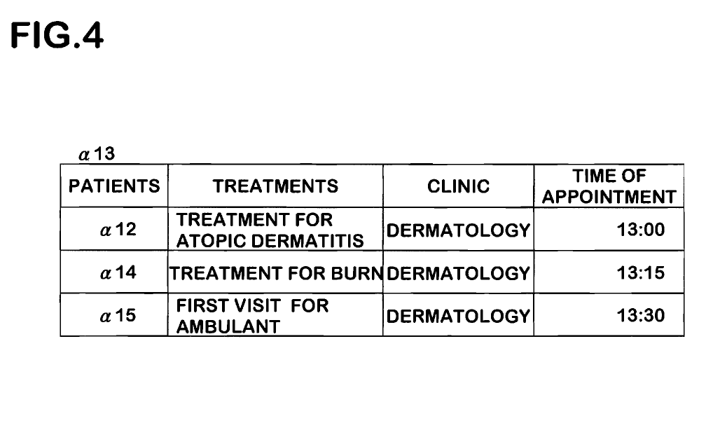

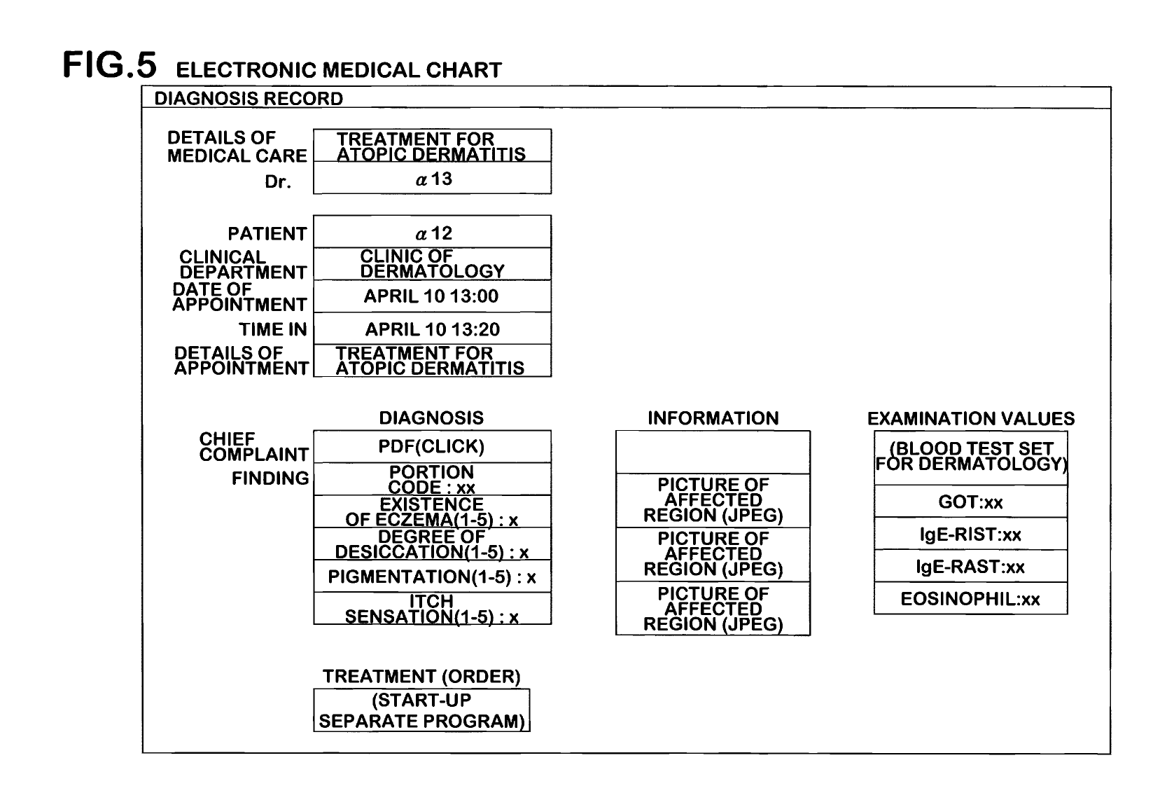

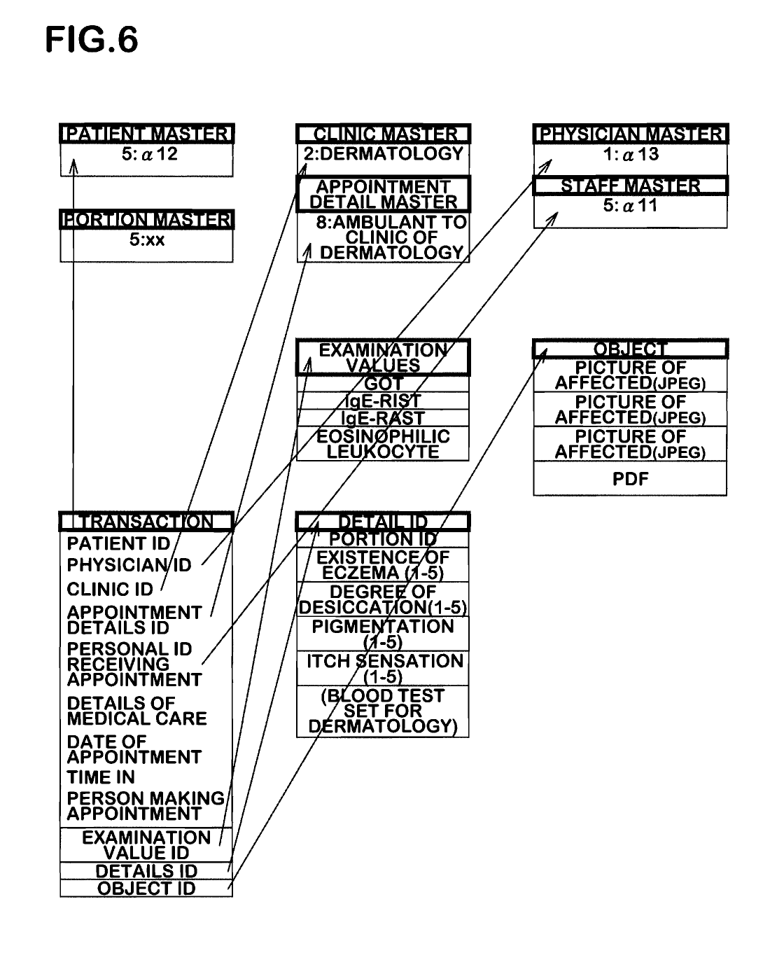

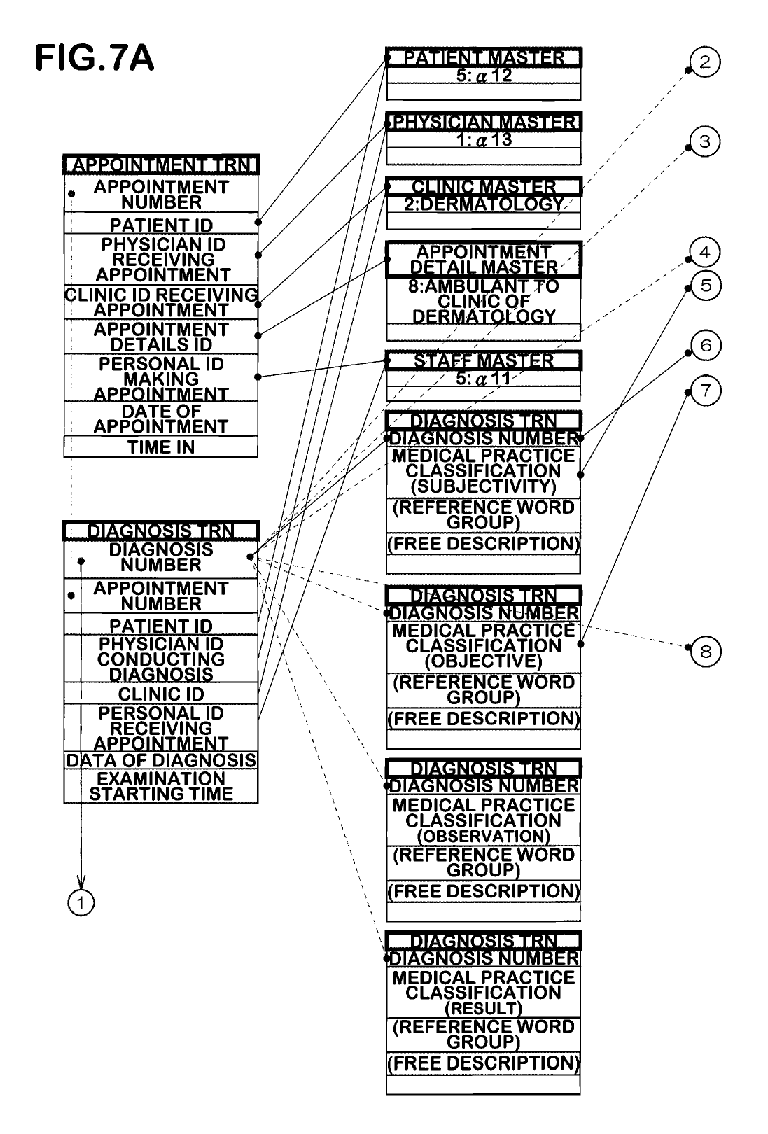

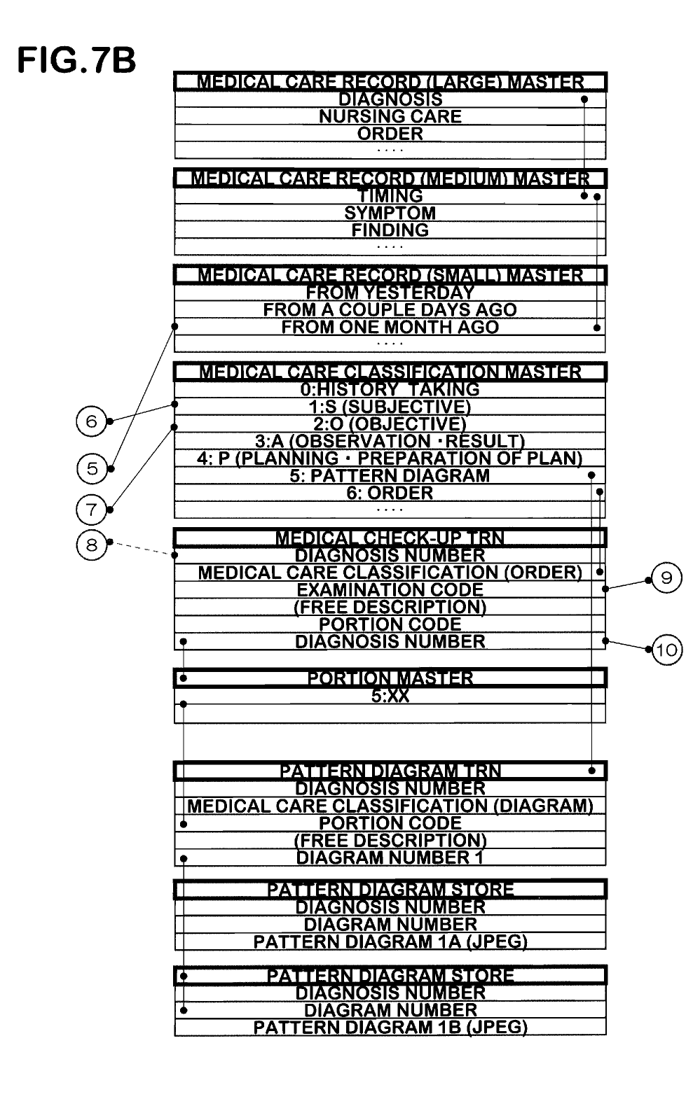

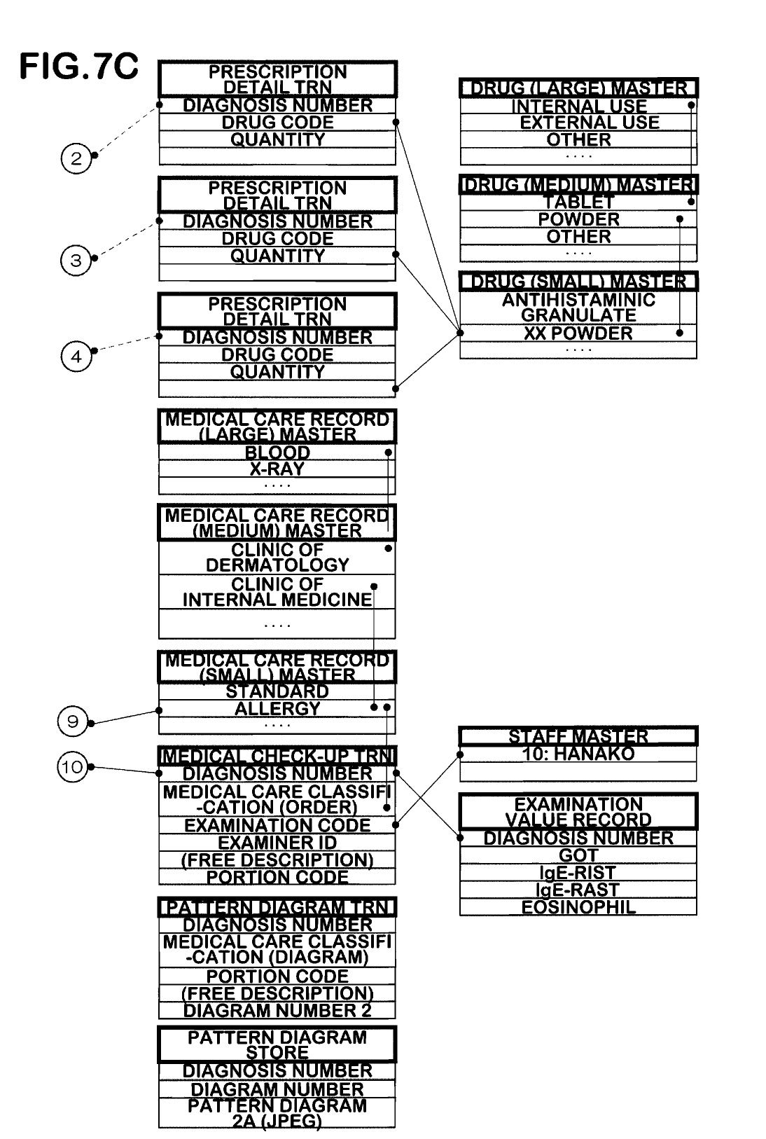

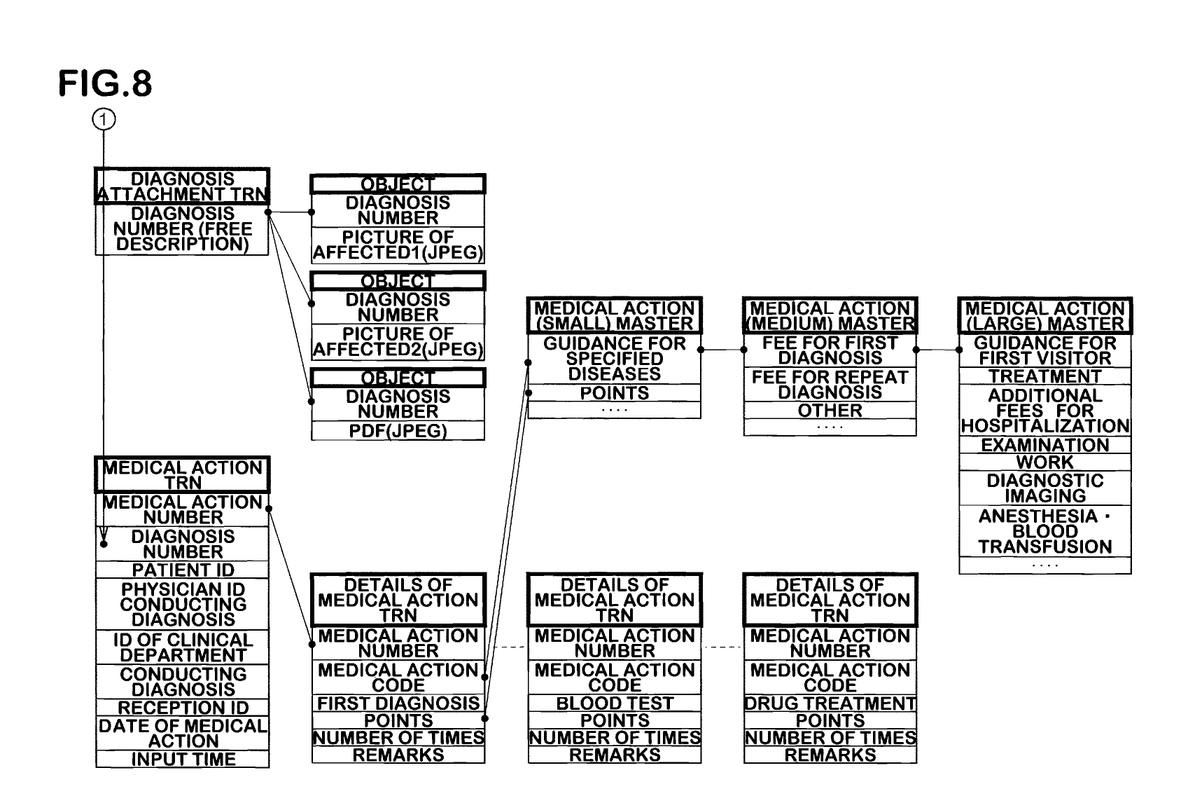

In the above-described embodiment, a CPU is used for realizing shown in FIG. 1 with programs. However, part or entirety of the functions may also be realized with hardware such as logic circuit and so on.

The second computer described in the above may also substitute with mobile phone units, mobile terminal and so on because the capability of the second computer is enough to perform various processing.

Alternatively, a part of processing of the programs may be performed by the operating system (OS).

What is claimed is:

1. A program for supporting work comprising:
A) a database storing instructions at each of phases for works, the database being characterized by the following a1) through a3);
a1) all the instructions being specified by one kind of action frame table including plurality of field items,
a2) labels of the field items being at least worker for the instructions, object person receiving works, supervisor for the worker, supervisor for the object person, time and place, and
a3) each of the instructions being specified by the action frame table, the each instruction corresponding to a detail table capable of listing data number without any limitation, the detail table storing operating actions performed by the worker,
B) an extraction program for extracting relevant instruction once a search formula specifying the worker is provided for the each of the instructions;
C) a display program for displaying the extracted instruction in a predetermined format;
D) a writing program for generating new instruction by writing data on the action frame table;
E) a correspondence program corresponding to information for specifying display format on each of the instructions; and
F) an instruction master generation program for generating an instruction master in which new instruction generated from the writing program is compared with instruction belong to upstream of the new instruction, an item being recognized as succession of such item when the same data exist in the item, another item being recognized as succession of another item representing duplication of data for the item when the same data exist in another item, and other items except for these defined in the above are recognized as empty items for inputting therein for each time.

2. A program for supporting work comprising:
A) a database storing instructions at each of phases for works, the database being characterized by the following a1) through a3);
a1) all the instructions being specified by one kind of action frame table including plurality of field items,
a2) the field items being at least worker for the instructions and object person receiving works, and
a3) each of the instructions being specified by the action frame table, the each instruction corresponding to a detail table capable of listing data number without any limitation,
B) an extraction program for extracting relevant instruction once a search formula specifying the field items is provided for each of the instructions; and
C) a display program for displaying the extracted instruction in a predetermined format.

3. The program for supporting work according to claim 2, wherein instruction for subsequent order linked by the instruction is written on the detail table.

4. The program for supporting work according to claim 3, wherein details of work performed by the instruction is written together with the subsequent order on the detail table.

5. The program for supporting work according to claim 4, wherein the detail table has a region for storing work result on the details of work together with the instruction for subsequent order.

6. The program for supporting work according to claim 2 further comprising supervisor for the worker, and supervisor for the object person as field items of the action frame table.

7. The program for supporting work according to claim 2, wherein title for display corresponding to each field item is stored in a correspondence table for each instruction during display of the field items in a predetermined format.

8. A system for supporting work comprising:
A) a database storing instructions at each of phases for works, the database being characterized by the following a1) through a3);
a1) all the instructions being specified by one kind of action frame table including plurality of field items,
a2) the field items being at least worker for the instructions and object person receiving works, and
a3) each of the instructions being specified by the action frame table, the each instruction corresponding to a detail table capable of listing data number without any limitation,
B) extraction means for extracting relevant instruction once a search formula specifying the field items is provided for each of the instructions; and
C) display control means for displaying the extracted instruction on display means in a predetermined format.

9. A database management system comprising:
A) a database storing instructions at each of phases for works, the database being characterized by the following a1) through a3);
a1) all the instructions being specified by one kind of action frame table including plurality of field items,
a2) the field items being at least worker for the instructions and object person receiving works, and
a3) each of the instructions being specified by the action frame table, the each instruction corresponding to a detail table capable of listing data number without any limitation.

12. A method of supporting work using a computer, the method comprising the steps of:
storing instructions at each of phases for works and recording data in a database characterized by the following a1) through a3);
a1) all the instructions being specified by one kind of action frame table including plurality of field items,
a2) the field items being at least worker for the instructions and object person receiving works, and
a3) each of the instructions being specified by the action frame table, the each instruction corresponding to a detail table capable of listing data number without any limitation,
B) extracting relevant instruction once a search formula specifying the field items being provided for each of the instructions; and
C) displaying the extracted instruction in a predetermined format.

11. A system for supporting work having a first computer storing instructions at each of phases for works and a second computer comprising:
A) the first computer being a database management device storing instructions at each of phases for works, the database being characterized by the following a1) through a3);
a1) all the instructions being specified by one kind of action frame table including plurality of field items,
a2) the field items being at least worker for the instructions and object person receiving works,
a3) each of the instructions being specified by the action frame table, the each instruction corresponding to a detail table capable of listing data number without any limitation, and
B) the second computer providing to the first computer a search formula specifying the field items and displaying the extracted instruction in a predetermined format once result of extraction is provided from the first computer.

12. The system for supporting work according to claim 11, wherein the second computer provides a search formula which recognize a person as worker, the person logging in thereto when the login process thereto is performed.
